(19) United States
(12) Patent Application Publication
(10) Pub. No.: US 2020/0197439 A1
(43) Pub. Date: Jun. 25, 2020

(54) IMMUNOTHERAPY FOR POLYOMAVIRUSES

(71) Applicants: Atara Biotherapeutics, Inc., South San Francisco, CA (US); The Council of the Queensland Institute of Medical Research, Herston (AU)

(72) Inventors: Rajiv Khanna, Herston (AU); George Robin Ambalathinga Thomas, St. Lucia (AU); Blake Tolu Aftab, Thousand Oaks, CA (US)

(21) Appl. No.: 16/331,414

(22) PCT Filed: Sep. 8, 2017
(86) PCT No.: PCT/US2017/050686

§ 371 Date: Mar. 7, 2019

Related U.S. Application Data
(60) Provisional application No. 62/385,456, filed on Sep. 9, 2016.

(51) Int. Cl. A61K 35/17; A61K 39/295

(52) U.S. Cl. CPC A61K 35/17; A61K 2039/5158; A61K 39/295

(57) ABSTRACT

Provided herein are methods and compositions related to polyomavirus epitopes useful in the treatment of cancer or a polyomavirus infection.

### RELATED APPLICATIONS

This application is a § 371 national-stage application based on PCT/US17/50686, filed Sep. 8, 2017, which claims the benefit of priority to U.S. Provisional Patent Application Ser. No. 62/385,456, filed Sep. 9, 2016, each of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 18, 2017, is named QAH-01325_SL.txt and is 80,299 bytes in size.

### BACKGROUND

Polyomaviruses are ubiquitous viruses that infect a wide range of mammalian species. Currently, more than 12 distinct human polyomavirus species have been identified, including BK polyomavirus (BKV), John Cunningham polyomavirus (JCV), and Merkel cell polyomavirus (MCV).

Most human polyomaviruses diseases are acquired in childhood, though clinically apparent diseases in immunocompetent hosts are generally rare. BKV and JCV viruses typically remain latent possibly in the lymphoid organs, neuronal tissue, and kidney. However, under the circumstances of immunosuppression, both JCV and BKV reactivate and may progress to significant organ disease. For example, BKV is urotheliotorpic and reactivation of BKV causes a form of interstitial nephritis, known as BK polyomaviruses associated nephropathy, which is typically associated with high graft loss when not recognized early. Neurotropic JC virus may enter the brain and cause progressive multifocal leukoencephalopathy, a demyelinating disease of the central nervous system with a high mortality rate. Various polyomaviruses have also been associated with different forms of cancer. For example, MCV has been associated with Merkel cell carcinoma, a rare but aggressive form of skin cancer. There are no known effective antiviral agents for treatment of polyomaviruses. Thus, new therapies are needed to treat and prevent polyomavirus infections and/or polyomavirus-associated cancer.

### SUMMARY

Provided herein are compositions and methods related to polyomavirus epitopes (e.g., epitopes listed in Tables 1, 2, 3, 4 and/or 5) that are recognized by T lymphocytes (e.g., cytotoxic T lymphocytes (CTLs) and/or helper T lymphocytes) and that are useful in the prevention and/or treatment of a polyomavirus infection (e.g., a BKV, JCV, or MCV virus infection), and/or cancer (e.g., a polyomavirus associated cancer, such as a BKV, JCV, or MCV associated cancer). In some embodiments, the compositions and methods relate to BKV epitopes (e.g., the epitopes listed in Table 1). In some embodiments, the compositions and methods provided herein relate to JCV epitopes (e.g., the epitopes listed in Table 2). In some embodiments, the compositions and methods relate to hybrid epitopes that incorporate sequence variations found within a viral strain and/or across related viral strains (e.g., the epitopes listed in Table 3).

In certain aspects, provided herein is a protein (e.g., an isolated protein) comprising one or more epitopes from one or more BKV antigens (e.g., epitopes from LTA, STA or VP1 viral antigens, such as the epitopes listed in Table 1), one or more JCV antigens ((e.g., epitopes from LTA, STA or VP1 viral antigens, such as the epitopes listed in Table 2) and/or one or more hybrid epitopes (e.g., the epitopes listed in Table 3). In some embodiments, the polypeptide comprises a plurality of such epitopes. In some embodiments, the polypeptide further comprises an intervening amino acid sequence between at least two of the plurality of epitopes. In some embodiments, the protein is capable of eliciting an immune response upon administration to a subject (e.g., a mammalian subject, such as a human subject).

In some embodiments, the epitopes are selected to provide broad coverage of the human population. In some embodiments, the epitopes have HLA class I restrictions to HLA-A1, -A2, -A3, -A11, -A23, -A24, -A26, -A29, -A30, -B7, -B8, -B27, -B35, -B38, -B40, -B41, -B44, -B51, -B56, -B57 or -B58. In some embodiments, the epitopes have HLA class II restrictions to HLA-DP, -DM, -DOA, -DOB, -DQ, or -DR. In some embodiments, the epitopes have HLA class II restrictions to HLA-DRB or -DQB. In some embodiments, the protein comprises, consists essentially of or consists of epitope amino acid sequences set forth in SEQ ID NOS: 5, 6, 36, 41 and 42. In some embodiments, provided herein is a pharmaceutical composition comprising a protein provided herein.

In certain aspects, provided herein is a nucleic acid (e.g., an isolated nucleic acid) encoding a protein disclosed herein. In some embodiments, provided herein is an expression construct comprising such a nucleic acid. In some embodiments, provided herein is a host cell comprising such an expression construct. In certain aspects provided herein is a method of producing an isolated protein comprising expressing the isolated protein in the host cell of provided herein and at least partly purifying the isolated protein. In some embodiments, provided herein is a pharmaceutical composition comprising a nucleic acid provided herein.

In certain aspects, provided herein is a T lymphocyte (e.g., a an isolated T lymphocyte, a CD4+ T lymphocyte, a CD8+ T lymphocyte) comprising a T cell receptor (TCR) that specifically binds to an epitope described herein presented on an HLA (e.g., a class I HLA, a class II HLA). In certain embodiments, provided herein is a method of expanding BK virus-specific T lymphocytes for adoptive immunotherapy, including: (i) contacting one or more cells isolated from a subject, wherein the one or more cells comprise T lymphocytes, with an antigen presenting cell presenting an epitope provided herein; and (ii) culturing the one or more cells under conditions such that BK virus-specific T-lymphocytes are expanded from said one or more cells. In specific embodiments, culturing the one or more cells is performed in the presence of IL-21. In some embodiments, the cells are cultured in the presence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 ng/ml IL-21, In some embodiments, the cells are cultured in no more than 30, 35, 40, 45, 50, 60, 70, 80, 90 or 100 ng/ml IL-21. In some embodiments, the cells are cultured in 10-50, 20-40, 25-35 or about 30 ng/ml IL-21. In some embodiments, the cells are cultured in 30 ng/ml IL-21. In certain embodiments, compared to expansion in the absence of IL-21, expansion in the presence of IL-21 results in an increase in the ratio of absolute number of polyomavirus-specific CD8 T cells to the absolute number of polyomavirus-specific CD4 T cells in the expanded population of T lymphocytes.

In certain embodiments, provided herein is a method of treating or preventing a polyomavirus infection (e.g., a BKV, JCV or MCV infection) and/or treating a polyomavirus-associated cancer (e.g., a BKV-associated, JCV-associated or MCV-associated cancer) and/or inducing a T-lymphocyte immune response in a subject comprising administering to the subject a protein, nucleic acid, T cell or pharmaceutical composition provided herein. In some embodiments, the subject is a mammal. In some embodiments, the subject is a human. In some embodiments, the subject is immunocompromised.

In certain aspects, provided herein is a method of detecting a BK virus infection in a subject, the method comprising detecting the presence of BKV-specific T lymphocytes by contacting T lymphocytes isolated from the subject with the isolated protein provided herein. In some embodiments, the method further comprising treating the BK virus infection in the subject according to a method described herein. In some embodiments, the subject is a mammal. In some embodiments, the subject is a human. In some embodiments, the subject is immunocompromised.

In certain aspects, provided herein are methods of treating a cancer in a subject (e.g., a polyomavirus-associated cancer, such as a BKV-, JCV-, or MCV-associated cancer). In some embodiments, the method comprises administering to the subject a pharmaceutical composition comprising cytotoxic T cells (CTLs) comprising T cell receptors (TCRs) that recognize one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the subject expresses a human leukocyte antigen (HLA) to which the one or more epitopes is restricted. In some embodiments, the CTLs are autologous to the subject. In some embodiments, the CTLs are not autologous to the subject. In some embodiments, the CTLs are obtained from a CTL library or bank. In some embodiments, the method comprises administering to the subject a vaccine composition comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the method comprises administering to the subject a pharmaceutical composition antigen presenting cells (APCs) presenting one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the subject expresses a human leukocyte antigen (HLA) to which the one or more epitopes is restricted.

In certain aspects, provided herein are methods of treating a polyomavirus infection (e.g. a BKV, MCV, or JCV infection) in a subject. In some embodiments the subject is immunocompromised. In some embodiments, the method comprises administering to the subject a pharmaceutical composition comprising CTLs comprising TCRs that recognize one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the subject expresses a HLA to which the one or more epitopes is restricted. In some embodiments, the CTLs are autologous to the subject. In some embodiments, the CTLs are not autologous to the subject. In some embodiments, the CTLs are obtained from a CTL library or bank. In some embodiments, the method comprises administering to the subject a vaccine composition comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the method comprises administering to the subject a pharmaceutical composition antigen presenting cells (APCs) presenting one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the subject expresses human leukocyte antigens (HLA) to which the one or more epitopes is restricted.

In some aspects, provided herein is a population of CTLs comprising T cell receptors (TCRs) that recognize one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3.

In some aspects, provided herein is a population of APCs presenting one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the APCs comprise B cells, antigen-presenting T cells, dendritic cells and/or artificial antigen-presenting cells, such as aK562 cells. In some aspects, the antigen-presenting cells (e.g., aK562 cells) express CD80, CD83, 41BB-L, and/or CD86. In some embodiments, provided herein are methods of treating or preventing cancer (e.g., a polyomavirus associated cancer, such as a BKV, JCV, or MCV associated cancer) and/or a polyomavirus (e.g., BKV, JVK, or MCV) infection in a subject comprising administering the APCs described herein to a subject.

In some aspects, provided herein is a polypeptide comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In certain aspects, provided herein is a nucleic acid molecule (e.g., a DNA molecule or an RNA molecule) encoding a polypeptide comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the nucleic acid molecule is a vector (e.g., an adenoviral vector). In some embodiments, provided herein are vaccine compositions comprising a polypeptide and/or a nucleic acid molecule described herein.

In some embodiments, provided herein are methods of generating, activating and/or inducing proliferation of polyomavirus-specific CTLs (e.g., BKV specific or JCV specific CTLs) comprising contacting CTLs with APCs that present one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3. In some embodiments, the CTLs are contacted with APCs in vitro. In some embodiments, the APCs comprise B cells, antigen-presenting T cells, dendritic cells and/or artificial antigen-presenting cells, such as aK562 cells. In some aspects, the antigen-presenting cells (e.g., aK562 cells) express CD80, CD83, 41BB-L, and/or CD86. In some embodiments, the CTLs are contacted to the APCs in the presence of one or more cytokines.

In some embodiments, provided herein are methods of generating APCs that present epitopes provided herein comprising contacting APCs with a polypeptide comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3 and/or a nucleic acid encoding a polypeptide comprising one or more (e.g., at least 1, 2, 3, 4, 5, 6, 7, 8, 9, 10, 11, 12, 13, 14, 15, 16, 17, 18. 19, 20, 21, 22, 23, 24, 25, 26, 27, 28, 29, 30 or more) of the epitopes listed in Table 1, Table 2 and/or Table 3 . In some embodiments, the APCs express HLA to which the one or more epitopes is restricted.

In some embodiments, the one or more epitopes comprise an epitope shared by two or more polyomaviruses. In some embodiments, the shared epitope comprises a region of sequence homology between the at least two polyomaviruses, and the region of sequence homology is at least 3, 4, 5, 6 or 7 amino acids across the full length of the epitope sequence. In some embodiments, the two polyomaviruses are BKV and JCV. In some embodiments, the at least three amino acids are LLL.

In other aspects, provided herein is a method of identifying a subject suitable for a method of treatment provided herein (e g , administration of CTLs, APCs, or vaccine compositions provided herein) comprising isolating a sample from the subject (e.g., a blood or tumor sample) and detecting the presence of an epitope provided herein, or a nucleic acid encoding an epitope provided herein. In certain embodiment, the subject is identified as suitable for a method of treatment provided herein if the subject expresses an HLA to which one or more of the epitopes described herein are restricted. In some embodiments, the subject identified as being suitable for a method of treatment provided herein is treated using the method of treatment.

### BRIEF DESCRIPTION OF THE DRAWINGS

FIG. 1 shows the in vitro expansion of BKV specific T cells. The dot blots show the detectable expression of IFN-γ by BKV specific T cells after growing the PBMCs with BKV antigens and CMV is shown as a positive control.

FIG. 2 shows the T cell response to BKV antigens. The graphs show the overall T cell response to BKV antigens in healthy individuals.

FIG. 3 shows peptide matrix for large T antigen (LTA), as well as the composition of the peptide pools following the matrix format.

FIG. 4 is a flow chart showing the process of epitope mapping described herein.

FIG. 5 has five panels and shows epitope mapping of HLA B*39 epitope. Panel A shows FACS blot for the CD8⁺ T cell response to STA OPP. Panel B shows STA pep pools 4 and 10 responded when overlayed on the matrix showed STA22 peptide to be the common peptide among the pools. ICS assay with ST22 stimulation showed a response which is shown in the FACS blot next to the matrix. Panel C shows fine epitope mapping by trimming the amino acids from either side of the ST22 peptide (SEQ ID NOS 127-139, respectively, in order of appearance). Panel D shows the responding peptides (SEQ ID NOS 127, 129-132, and 134-135, respectively, in order of appearance) from trimming process are titrated to see the most immunogenic section of the peptide which showed VHCPCMLCQL (SEQ ID NO: 8) to be the epitope sequence. Panel E shows antigen presentation assay using the peptide loaded HLA restricted LCLs showing the epitope to be HLA B*39 restricted.

FIG. 6 shows transcriptional regulators in BKV and CMV specific T cells. The histogram shows the comparison of CMV and BKV specific T cells for the expression of T bet, Eomes, Granzyme B and perforin. Histogram lines shows the expression in CMV specific T cells and BKV specific T cells as indicated.

FIG. 7 shows in vitro expansion of BKV-specific T cells following stimulation with pooled BKV epitopes (see Table 1). PBMC from healthy volunteers were stimulated with synthetic BKV peptides for 1 h and then cultured for 12-14 days in the presence different cytokine combinations. These included IL-2 (10 ng/ml), IL-21 (30 ng/ml), IL7 (10 ng/ml), IL12 (10 ng/ml) and/or IL15 (10 ng/ml). BKV specificity of these T cells was assessed using standard intracellular cytokine assays.

FIG. 8 shows consensus sequence alignments between BKV and JCV LTA (SEQ ID NOS 140 and 141, respectively), STA (SEQ ID NOS 142 and 143, respectively) and VP1 (SEQ ID NOS 144 and 145, respectively) amino acid sequences.

FIG. 9 shows consensus sequence alignments between BKV and MCV LTA (SEQ ID NOS 146-153, respectively, in order of appearance), STA (SEQ ID NOS 154 and 155, respectively) and VP1 (SEQ ID NOS 156 and 157, respectively) amino acid sequences.

FIG. 10 shows the transcriptional factor and effector molecule profile of BKV specific T cells grown in the presence of IL2 or IL2 and IL21. The frequencies of granzyme high and T bet high cells were higher in cells grown in the presence of IL-2 and IL-21.

FIG. 11 shows the IFN-γ expression of CD4 and CD8 T cells grown in the presence of IL-2 or IL2 and IL-21 and analysed for the specificity using BKV epitopes.

FIG. 12 shows the number of CD4 and CD8 cells after culture in the presence of IL-2 or IL-2 and IL-21. The total number of BKV specific CD4+ T cells was reduced in the cultures grown in the presence of IL-2 and IL-21 compared to cultures grown in IL2 alone.

FIG. 13 shows that the percentage of CD25⁺ cells in both CD8⁺ and CD4⁺ T cell populations was higher in the T cells grown in the presence of IL-2 alone compared to cells grown in presence of IL-2 and IL-21.

FIG. 14 shows neuropilin1 expression on CD4⁺CD25^{hi}CD127^{low} cells (Treg cells).

FIG. 15 shows representative IFN-γ expression data from exemplary epitopes that show BKV/JCV cross-reactivity. Figure discloses SEQ ID NOS 14 and 57, respectively, in order of appearance.

FIG. 16 shows representative IFN-γ expression data from cells expanded using a JCV epitope and recalled using various concentrations of either the JCV epitope or the corresponding BKV epitope.

### DETAILED DESCRIPTION

### General

Provided herein are compositions and methods related to polyomavirus epitopes (e.g., epitopes listed in Tables 1, 2, 3, 4 and/or 5) that are recognized by T lymphocytes (e.g., cytotoxic T lymphocytes (CTLs) and/or helper T lymphocytes) and that are useful in the prevention and/or treatment of a polyomavirus infection (e.g., a BKV, JCV, or MCV virus infection), and/or cancer (e.g., a polyomavirus associated cancer, such as a BKV, JCV, or MCV associated cancer). In some embodiments, the compositions and methods relate to BKV epitopes (e.g., the epitopes listed in Table 1). In some embodiments, the compositions and methods provided herein relate to JCV epitopes (e.g., the epitopes listed in Table 2). In some embodiments, the compositions and methods relate to hybrids epitopes that encompass variations found within or across BKV and JCV epitopes (e.g., the epitopes listed in Table 3).

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen presenting cell provided herein and/or a CTL provided herein.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “binding” or “interacting” refers to an association, which may be a stable association, between two molecules, e.g., between a TCR and a peptide/HLA, due to, for example, electrostatic, hydrophobic, ionic and/or hydrogen-bond interactions under physiological conditions. A TCR “recognizes” a T cell epitope that it is capable of binding to when the epitope is presented on an appropriate HLA.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

As used herein, the term “cancer” includes, but is not limited to, solid tumors and blood borne tumors. The term cancer includes diseases of the skin, tissues, organs, bone, cartilage, blood and vessels. The term “cancer” further encompasses primary and metastatic cancers.

The term “homologous” as used herein, refers to sequence similarity (e.g., a nucleic acid or amino acid sequence) between two regions of the same sequence strand or between regions of two different sequence strands. The term “homologous” may also be used to refer to sequence similarity between two regions of the same sequence strand or between regions of two different sequence strands. For example, when an amino acid residue position in both regions is occupied by the same amino acid residue, then the regions are homologous at that position. A first region is homologous to a second region if at least one nucleotide residue position of each region is occupied by the same residue. Homology between two regions is expressed in terms of the proportion of nucleotide or amino acid residue positions of the two regions that are occupied by the same nucleotide or amino acid residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “isolated” refers to material that has been removed from its natural state or otherwise been subjected to human manipulation. Isolated material may be substantially or essentially free from components that normally accompany it in its natural state, or may be manipulated so as to be in an artificial state together with components that normally accompany it in its natural state.

The term “peptide” refers to a peptide or polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “epitope” means a protein determinant capable of specific binding to an antibody or TCR. Epitopes usually consist of chemically active surface groupings of molecules such as amino acids or sugar side chains. Certain epitopes can be defined by a particular sequence of amino acids to which an antibody is capable of binding.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of an antibody to bind to a predetermined antigen or the ability of a peptide to bind to its predetermined binding partner. Typically, an antibody or peptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Epitopes

In certain embodiments provided herein are methods and compositions related BKV epitopes, JCV epitopes, MCV epitopes and/or epitopes that comprise sequences homologous between BKV, JCV and/or MCV epitopes that are recognized CTLs when presented on an HLA. In certain embodiments, the epitopes described herein are useful in the prevention and/or treatment of a polyomavirus infection (e.g., a BKV, JCV, or MCV viral infections) and/or cancer (e.g., a polyomavirus associated cancer expressing an epitope provided herein) and/or for the generation of pharmaceutical agents (e.g., CTLs and/or APCs) that are useful in the prevention and/or treatment of a polyomavirus infection (e.g., a BKV, JCV, or MCV viral infections) and/or cancer (e.g., a polyomavirus associated cancer expressing an epitope provided herein). In certain embodiments, the epitope is a BKV epitope listed in Table 1, and/or a JCV epitope listed in Table 2. In some embodiments, the epitope is a hybrid epitope comprising amino acids from both a BKV epitope and a homologous JCV epitope and/or amino acid variants found within different BKV or JCV [insert appropriate noun here]. Exemplary hybrid epitopes are listed in Table 3. In some embodiments, the compositions and methods provided herein further comprise an MCV epitope (e.g., a MCV epitope homologous to an epitope listed in Tables 1-3). In some embodiments, the compositions and methods described herein further relate to epitopes from addition viruses, such as EBV, CMV, or ADV. In some embodiments, the epitopes are HLA class I-restricted T cell epitopes. In other embodiments, the epitopes are HLA class II-restricted T cell epitopes.

TABLE 1
Exemplary BKV HLA class I and class
II-restricted T cell epitopes

| | | HLA | SEQ |
|---|---|---|---|
| Epitope | Antigen | Restriction | ID NO.: |
| DSQHSTPPK | LTA | A*11 | 1 |
| AVDTVLAKK | LTA | A*11 | 2 |
| CYCIDCFTQ | STA | A*24 | 3 |
| LPLMRKAYL | LTA/STA | B*07/B*08 | 4 |
| FPLCPDTLY | STA | B*35 | 5 |
| TLYCKEWPI | STA | B*35 | 6 |
| EPL(V/G)W(K/I)DCY | STA | B*35 | 7 |
| VHCPCMLCQL | STA | B*39 | 8 |
| NREESMELMDL | LTA/STA | B*40 | 9 |
| MELMDLLGL | LTA/STA | B*40 | 10 |
| FFAVGGDPLEM | STA | B*40 | 11 |
| YCIDCFT(Q/E)W | STA | B*57 | 12 |
| TPHRHRVSA | LTA | B*56 | 13 |
| LLLGMYLEF | LTA | A*29 | 14 |
| V(F/L)LLLGMYLEF | LTA | A*23 | 15 |
| IEESI(Q/H)GGL | LTA | B*40 | 16 |
| TEV(I/M)GITSML | VP1 | B*40 | 17 |
| ARIPLPNL | VP1 | B*27 | 18 |
| VKNPYPISFLL | VP1 | Cw*07 | 19 |
| QAVDTVLAKK | LTA | A*11 | 20 |
| MLT(E/D)RFNHIL | LTA | A*02 | 21 |
| LLLIWFRPV | LTA | A*02:01 | 22 |
| AIT(E/Q)VECFL | VP1 | A*02:01 | 23 |
| (R/K)LDSEISMY | LTA | A*01 | 24 |
| SVKVNLEKH | LTA | A*03 | 25 |
| AYLR(K/R)CKEF | LTA | A*24 | 26 |
| (N)ILMWEAVTL | VP1 | A*02 | 27 |
| LPGDPDMIRYIDRQG | VP1 | A24/A29/B7/B39 | 28 |
| LEVKTGVDAITEVEC | VP1 | A24/A29/B7/B39 | 29 |
| DICGLF(T/I)NSSGTQQW | VP1 | A24/A29/B7/B39 | 30 |
| ESQVEEVRVFDGTEQ | VP1 | A24/A29/B7/B39 | 31 |
| GTQQWRGLARYFKIR | VP1 | DRB1*11/8 | 32 |
| RGLARYFKIRLRKRS | VP1 | DRB1*11 | 33 |
| RKAYLRKCKEFHPDK | LTA | DRB1*13 | 34 |
| WDEDLFCHEDMFASD | LTA | DQB5*01 | 35 |
| CFTQWFGLDLTEETL | STA | DRB1*03/04 | 36 |
| GGDEDKMKRMNTLYK | LTA/STA | DRB1*13 | 37 |
| KMKRMNTLYKKMEQD | LTA/STA | DRB1*13 | 38 |
| FNVPKRRYWLFKGPI | LTA | DRB1*15 | 39 |
| RRYWLFKGPIDSGKT | LTA | DRB1*15 | 40 |
| VGPLCKADSLYVSAA | VP1 | ND* | 41 |
| AYLDKNNAYPVECWI | VP1 | ND* | 42 |
| DMIRYIDRQGQLQTK | VP1 | ND* | 43 |
| SQHSTPPKK | LTA | A*11 | 121 |
| FPLCPDTLYC | STA | B*35 | 122 |
| LLIKGGVEV | ND* | ND* | 123 |

*ND: Not defined

TABLE 2
Exemplary epitope sequences from JCV
homologous to BKV epitope sequences

| | | HLA | SEQ |
|---|---|---|---|
| Epitope* | Antigen | Restriction | ID NO.: |
| KS(Q/R)HSTPP(K/R)K | LTA | A*11 | 44 |
| AVDTVAAKQ | LTA | A*11 | 45 |
| CYCFDCFRQ | STA | A*24 | 46 |
| IPVMRKAYL | LTA/STA | B*07/B*08 | 47 |
| FPPNSDTLY | STA | B*35 | 48 |
| FLYCKEWPN | STA | B*35 | 49 |
| SPLV(W/R)IDCY | STA | B*35 | 50 |
| VHCPCLMCML | STA | B*39 | 51 |
| NREESMELMDLL | LTA/STA | B*40 | 52 |
| MELMDLLGL | LTA/STA | B*40 | 53 |
| FFSVGGEALEL | VP1 | B*40 | 54 |
| YCFDCFRQW | STA | B*57 | 55 |
| TPHRHRVSA | LTA | B*56 | 56 |
| LLMGMYLDF | LTA | A*29 | 57 |
| VFLLMGMYLDF | LTA | A*23 | 58 |
| VE(E/G)SIQGGL | LTA | B*40 | 59 |
| TEV(I/L)GVTLMN | VP1 | B*40 | 60 |
| ARIPLPNLN | VP1 | B*27 | 61 |
| VKNPYPISFLL | VP1 | Cw*07 | 62 |
| LPGDPDMMRYVDKYG | VP1 | HLA A24/A29/ | 63 |
| | | B7/B39 | |
| LEVKTGVDSITEVEC | VP1 | HLA A24/A29/ | 64 |
| | | B7/B39 | |
| DVCGMFTNRSGSQQW | VP1 | HLA A24/A29/ | 65 |
| | | B7/B39 | |
| DAQVEEVRVFEGTEE | VP1 | HLA A24/A29/ | 66 |
| | | B7/B39 | |
| QAVDTVAAKQ | LTA | A*11 | 67 |
| ML(V/M)(E/Q/G)RFNFLL | LTA | A*02 | 68 |
| LLLIWFRPV | LTA | A*02:01 | 69 |
| S(I/V)TEVECFL | VP1 | A*02:01 | 70 |
| RLDLEISMY | LTA | A*01 | 71 |
| SV(K/R)VNLERKH | LTA | A*03 | 72 |
| AYLK(K/R)CKEL | LTA | A*24 | 73 |
| (N)LLMWEAVTV | VP1 | A*02 | 74 |
| GSQQWRGLSRYFKVQ | VP1 | DRB1*11/8 | 75 |
| RGLSRYFKVQLRKRR | LTA | DRB1*11/8 | 76 |
| RKAYLKKCKELHPDK | LTA | DRB1*13 | 77 |
| WDEDLFCHEEMFASD | LTA | DQB5*01 | 78 |
| CFRQWFGCDLTQEAL | LTA/STA | DRB1*03/04 | 79 |
| GGDEDKMKRMNFLYK | LTA | DRB1*13 | 80 |
| KMKRMNFLYKKMEQG | VP1 | DRB1*13 | 81 |
| LNIPKKRYWLFKGPIDSGKT | VP1 | DRB1*15 | 82 |
| KRYWLFKGPIDSGKT | VP1 | DRB1*15 | 83 |
| VGPLCKGDNLYLSAV | VP1 | ND** | 84 |
| AYLDKNKAYPVECWV | VP1 | ND** | 85 |
| DMMRYVDRYGQLQTK | VP1 | ND** | 86 |
| SQHSTPPKK | LTA | A*11 | 124 |
| FPPNSDTLYC | STA | B*35 | 125 |
| LLIKGGVEV | ND* | ND* | 126 |

*Amino acid residues which are variant from the BKV
epitope are bolded and underlined.
**Not defined

TABLE 3
Exemplary epitope sequences from JCV/BKV hybrid epitope sequences

| Epitope* | Antigen | HLA Restriction | SEQ ID NO: |
|---|---|---|---|
| (D/K)S(Q/K)HSTPP(K/R/KK) | LTA | A*11 | 87 |
| AVDTV(L/A)AK(K/Q) | LTA | A*11 | 88 |
| CYC(I/F)DCF(T/R)Q | STA | A*24 | 89 |
| (L/I)P(L/V)MRKAYL | LTA/STA | B*07/B*08 | 90 |
| FP(L/P)(P/N)(P/S)DTLY | STA | B*35 | 91 |
| (F/T)LYCKEWP(I/N) | STA | B*35 | 92 |
| (E/S)PL(VWI/VWK/VRI/GWI)DCY | STA | B*35 | 93 |
| VHCPC(M/L)(L/M)C(M/Q)L | STA | B*39 | 94 |
| YC(I/F)DCF(T/R)(Q/E)W | STA | B*57 | 95 |
| LL(L/M)GMYL(E/D)F | LTA | A*29 | 96 |
| V(F/L)LLMGMYL(E/D)F | LTA | A*23 | 97 |
| (I/V)E(E/G)SI(Q/H)GGL | LTA | B*40 | 98 |
| TEV(I/M/L)G(I/V)T(S/L)M(L/N) | VP1 | B*40 | 99 |
| LPGDPDM(I/M)RY(I/V)D(R/K)(Q/Y)G | VP1 | HLA A24/A29/B7/B39 | 100 |
| D(I/V)CG(L/M)F(T/I)N(S/R)SG(T/S)QQW | VP1 | HLA A24/A29/B7/B39 | 101 |
| QAVDTV(L/A)AK(K/Q) | LTA | A*11 | 102 |
| ML(T/V/M)(E/D/Q)RFN(H/F)(I/L)L | LTA | A*02 | 103 |
| (AI/SI/SV)T(E/Q)VECFL | VP1 | A*02:01 | 104 |
| (R/K)LD(S/L)EISMY | LTA | A*01 | 105 |
| SV(K/R)VNLE(E/R)KH | LTA | A*03 | 106 |
| AYL(R/K)KCKE(F/L) | LTA | A*24 | 107 |
| (N)(I/V)MWEAVT(L/V) | VP1 | A*02 | 108 |
| G(T/S)QQWRGL(A/S)RYFK(I/V)(R/Q) | VP1 | DRB1*11/8 | 109 |
| RGL(A/S)RYFK(I/V)(R/Q)LRKR(S/R) | LTA | DRB1*11/8 | 110 |
| RKAYL(RR/RK/KK)CKE(F/L)HPDK | LTA | DRB1*13 | 111 |
| WDEDLFCHE(D/E)MFASD | LTA | DQB5*01 | 112 |
| CF(T/R)QWFG(L/C)DLT(E/Q)E(T/A)L | LTA/STA | DRB1*03/04 | 113 |
| GGDEDKMKRMN(T/F)LYK | LTA | DRB1*13 | 114 |
| KMKRMN(T/F)LYKKMEQ(D/G) | VP1 | DRB1*13 | 115 |
| (F/L)N(V/I)PK(R/K)RYWLFKGPIDSGKT | VP1 | DRB1*15 | 116 |
| (R/K)RYWLFKGPIDSGKT | VP1 | DRB1*15 | 117 |
| VGPLCK(A/G)D(S/N)LYLSAV | VP1 | ND** | 118 |
| AYLDKN(N/K)AYPVECW(I/V) | VP1 | ND** | 119 |
| DM(I/M)RY(I/V)DR(Q/G)GQLQTK | VP1 | ND** | 120 |

**Not defined

In some embodiments, provided herein are peptides comprising one or more of the epitopes from Table 1, Table 2 and/or Table 3. In some embodiments, the peptides disclosed herein are full length viral proteins (e.g., full length BKV, JCV and/or MCV proteins). In some embodiments, the peptide is not a full-length viral protein (e.g., not a full length BKV, JCV and/or MCV protein). In some embodiments, the peptides disclosed herein comprise BKV and JCV epitopes with sequence homology (e.g., epitopes listed in Tables 1-3). In some embodiments, the peptides disclosed herein comprise less than 100, 90, 80, 70, 60, 50, 40, 30, 25, 20, 15 or 10 contiguous amino acids of a viral protein. In some embodiments, the peptides disclosed herein comprise two or more of the epitopes listed in Table 1, Table 2 and/or Table 3. For example, in some embodiments, the peptide disclosed herein comprises two or more of the epitopes listed in Table 1, Table 2 and/or Table 3 connected by polypeptide linkers. In some embodiments, the peptide provided herein comprises at least 1, 2, 3, 4, 5, 6, 7, 8, 9, 10, 11, 12, 13, 14, 15, 16, 17, 18, 19, 20, 21, 22, 23, 24, 25, 26, or 27 epitopes (e.g., at least 1, 2, 3, 4, 5, 6, 7, 8, 9, 10, 11, 12, 13, 14, 15, 16, 17, 18, 19, 20, 21, 22, 23, 24, 25, 26, or 27 of the epitopes listed in Table 1, Table 2 and/or Table 3).

In certain aspects, provided herein is a polypeptide and/or protein (e.g., an isolated polypeptide or protein) comprising a plurality of epitopes from one or more BKV or JCV antigens (e.g., epitopes from LTA, STA or VP1 viral antigens, such as the epitopes listed in Tables 1, 2 or 3). In some embodiments, the polypeptide or protein further comprises an intervening amino acid sequence between at least two of the plurality of epitopes. In some embodiments, the intervening amino acids or amino acid sequences are proteasome liberation amino acids or amino acid sequences. Non-limiting examples of proteasome liberation amino acids or amino acid sequences are or comprise AD, K or R. In some embodiments, the intervening amino acids or amino acid sequence are TAP recognition motifs. Typically, TAP recognition motifs may conform to the following formula: (R/N:I/Q:W/Y)ₙ where n is any integer≥1. Non-limiting examples of TAP recognition motifs include RIW, RQW, NIW and NQY. In some embodiments, the epitopes provided herein are linked or joined by the proteasome liberation amino acid sequence and, optionally, the TAP recognition motif at the carboxyl terminus of each epitope.

In some embodiments, the polypeptides provided herein further comprise epitopes from and at least one additional virus (e.g., Epstein Barr virus (EBV), cytomegalovirus (CMV), and/or adenovirus (ADV)). In some embodiments the peptides comprise epitopes two or more viruses. In some embodiments the peptides comprise epitopes three or more viruses. In some embodiments the peptides comprise epitopes four or more viruses. In some embodiments the peptides comprise epitopes five or more viruses. For example, in some embodiments the peptides comprise sequences from at least two, three, four or five of JCV, BKV, MCV, EBV, CMV and/or ADV.

In some embodiments, provided herein is a polyepitope protein (i.e., a single chain of amino acid residues comprising multiple T cell epitopes not linked in nature) comprising two or more of the epitopes described herein. In some embodiments, the T cell epitopes in the polyepitope protein are connected via an amino acid linker. In some embodiments, the T cell epitopes in the polyepitope protein are directly linked without intervening amino acids. Examples of polyepitope proteins, methods of generating polyepitope proteins, and vectors encoding polyepitope proteins can be found in Dasari et al., Molecular Therapy—Methods & Clinical Development (2016) 3, 16058, which is hereby incorporated by reference in its entirety.

In some embodiments, the compositions and methods provided herein comprise or relate to naturally occurring variants of the epitopes listed in Tables 1 and/or 2. For example, in some embodiments, provided herein is a polyepitope protein that comprises two or more (e.g., at least 3, 4, 5, 6, 7, 8, 9 or 10) naturally occurring variants of an epitope listed in Table 1 and/or Table 2.

In some embodiments, the sequence of the epitopes provided herein have a sequence disclosed herein except for 1 or more (e.g., 1, 2, 3, 4, 5, 6, 7, 8, 9, 10 or more) conservative sequence modifications. As used herein, the term “conservative sequence modifications” is intended to refer to amino acid modifications that do not significantly affect or alter the interaction between a TCR and a peptide containing the amino acid sequence presented on an HLA. Such conservative modifications include amino acid substitutions, additions (e.g., additions of amino acids to the N or C terminus of the peptide) and deletions (e.g., deletions of amino acids from the N or C terminus of the peptide). Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the peptides described herein can be replaced with other amino acid residues from the same side chain family and the altered peptide can be tested for retention of TCR binding using methods known in the art. Modifications can be introduced into an antibody by standard techniques known in the art, such as site-directed mutagenesis and PCR-mediated mutagenesis.

In some aspects, provided herein are cells that present one or more peptide described herein (e.g., a peptide comprising an epitope listed in Table 1, Table 2 and/or Table 3). In some embodiments, the cell is a mammalian cell. In some embodiments the cell is an antigen-presenting cell (APC) (e.g., an antigen-presenting T-cell, a dendritic cell, a B cell, a macrophage or am artificial antigen-presenting cell, such as aK562 cell). A cell presenting a peptide described herein can be produced by standard techniques known in the art. For example, a cell may be pulsed to encourage peptide uptake. In some embodiments, the cells are transfected with a nucleic acid encoding a peptide provided herein. In some aspects, provided herein are methods of producing antigen-presenting cells (APCs), comprising pulsing a cell with the peptides described herein. Exemplary examples of producing antigen-presenting cells can be found in WO2013088114, hereby incorporated in its entirety.

The peptides provided herein can be isolated from cells or tissue sources by an appropriate purification scheme using standard protein purification techniques, can be produced by recombinant DNA techniques, and/or can be chemically synthesized using standard peptide synthesis techniques. The peptides described herein can be produced in prokaryotic or eukaryotic host cells by expression of nucleotides encoding a peptide(s) of the present invention. Alternatively, such peptides can be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Nucleic Acid Molecules

Provided herein are nucleic acid molecules that encode the epitopes and peptides described herein. The nucleic acids may be present, for example, in whole cells, in a cell lysate, or in a partially purified or substantially pure form. A nucleic acid molecule described herein can be isolated using standard molecular biology techniques and the sequence information provided herein. For example, oligonucleotides corresponding to the nucleotide sequence of one or more of the epitopes listed in Tables 1, 2, or 3 can be prepared by standard synthetic techniques, i.e., using an automated DNA synthesizer.

In some embodiments, provided herein are vectors (e.g., a viral vector, such as an adenovirus based expression vector) that contain the nucleic acid molecules described herein. A viral vector may contain additional DNA segments may be ligated into the viral genome. Certain vectors are capable of autonomous replication in a host cell into which they are introduced (e.g., bacterial vectors having a bacterial origin of replication, episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In some embodiments, provided herein are nucleic acids operable linked to one or more regulatory sequences (e.g., a promoter) in an expression vector. In some embodiments the cell transcribes the nucleic acid provided herein and thereby expresses an antibody, antigen binding fragment thereof or peptide described herein. The nucleic acid molecule can be integrated into the genome of the cell or it can be extrachromosomal.

In some embodiments, the nucleic acid vectors or recombinant adenoviruses provided herein encode one or more epitopes listed in Tables 1, 2, and/or 3. For example, the nucleic acid vectors or recombinant adenoviruses may consist of one or more epitopes from the same table (e.g., one or more epitopes from Table 1, one or more epitopes from Table 2, or one or more epitopes from Table 3). Or, the nucleic acid vectors or recombinant adenoviruses may consist of one or more epitopes from the same table (e.g., Table 1), and one or more epitopes from a different table (e.g., Table 2). In some embodiments, the nucleic acid vectors or recombinant adenoviruses provided herein encode for no more than 20, 19, 18, 17, 16, 15, 14, 13, 12, 11, 10, 9, 8, 7, 6, 5, 4, 3, 2 or 1 amino acids in addition to the epitopes listed in Tables 1, 2, or 3.

In some embodiments, the nucleic acid vectors comprise nucleic acid sequences that have undergone codon optimization. In such embodiments, a coding sequence is constructed by varying the codons in each nucleic acid used to assemble the coding sequence. In general, a method to identify a nucleotide sequence that optimizes codon usages for production of a peptide comprises at least the following steps (a) through (e). In step (a), oligomers are provided encoding portions of the polypeptide containing degenerate forms of the codon for an amino acid encoded in the portions, with the oligomers extended to provide flanking coding sequences with overlapping sequences. In step (b), the oligomers are treated to effect assembly of the coding sequence for the peptide. The reassembled peptide is included in an expression system that is operably linked to control sequences to effect its expression. In step (c), the expression system is transfected into a culture of compatible host cells. In step (d), the colonies obtained from the transformed host cells are tested for levels of production of the polypeptide. In step (e), at least one colony with the highest or a satisfactory production of the polypeptide is obtained from the expression system. The sequence of the portion of the expression system that encodes the protein is determined. Further description of codon optimization is provided in U.S. Patent Publication number US2010/035768, which is incorporated by reference in its entirety.

### Antigen Presenting Cells

In some aspects, provided herein are APCs that present (e.g., on HLA) one or more T cell epitopes provided herein (e.g., one or more T cell epitopes listed in Table 1, Table 2 and/or Table 3). In some embodiments, the HLA is a class I HLA. In some embodiments, the HLA is a class II HLA. In some embodiments, the class I HLA has an a chain polypeptide that is HLA-A, HLA-B, HLA-C, HLA-E, HLA-F, HLA-g, HLA-K or HLA-L. In some embodiment, the class II HLA has an α chain polypeptide that is HLA-DMA, HLA-DOA, HLA-DPA, HLA-DQA or HLA-DRA. In some embodiments, the class II MHLA has a β chain polypeptide that is HLA-DMB, HLA-DOB, HLA-DPB, HLA-DQB or HLA-DRB. In some embodiments, APCs present at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38, 39 T cell epitopes Table 1, Table 2 and/or Table 3).

In some embodiments, the APCs are B cells, antigen presenting T-cells, dendritic cells, or artificial antigen-presenting cells (e.g., aK562 cells). Dendritic cells for use in the process may be prepared by taking PBMCs from a patient sample and adhering them to plastic. Generally the monocyte population sticks and all other cells can be washed off. The adherent population is then differentiated with IL-4 and GM-CSF to produce monocyte derived dendritic cells. These cells may be matured by the addition of IL-1β, IL-6, PGE-1 and TNF-α (which upregulates the important co-stimulatory molecules on the surface of the dendritic cell) and are then contacted with a recombinant adenovirus described herein.

In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference.

In certain aspects, provided herein are methods of generating APCs that present the two or more of the T cell epitopes described herein comprising contacting an APC with a nucleic acid vector and/or recombinant adenoviruses encoding T cell epitopes described herein and/or with a polyepitope produced by the nucleic acid vectors or recombinant adenoviruses described herein. In some embodiments, the APCs are irradiated.

### T Cells

In certain aspects, provided herein are T cells and populations of T cells (e.g., CD4 T cells and/or CD8 T cells) that express a TCR (e.g., an αβ TCR or a γδ TCR) that recognize a peptide described herein (e.g., an epitope listed in Table 1, Table 2 and/or Table 3) presented on HLA. In some embodiments, the T cell is a CD8 T cell (a CTL) that expresses a TCR that recognizes a peptide described herein presented on a class I HLA. In some embodiments, the T cell is a CD4 T cell (a helper T cell) that recognizes a peptide described herein presented on a class II HLA.

In some aspects, provided herein are methods of generating, activating and/or inducing proliferation of T cells (e.g., CTLs) that recognize one or more of the epitopes described herein. In some embodiments, a sample comprising CTLs (i.e., a PBMC sample) is incubated in culture with an APC provided herein (e.g., an APC that presents a peptide comprising a BKV and/or JCV epitope described herein on a class I HLA complex). In some embodiments, the sample containing T cells are incubated 2 or more times with APCs provided herein. In some embodiments, the T cells are incubated with the APCs in the presence of at least one cytokine. In some embodiments, the cytokine is IL-4, IL-7 and/or IL-15. Exemplary methods for inducing proliferation of T cells using APCs are provided, for example, in U.S. Pat. Pub. No. 2015/0017723, which is hereby incorporated by reference.

In some aspects, provided herein is a population of CTLs collectively comprising T cell receptors that recognize one or more T cell epitopes (e.g., one or more of the T cell epitopes listed in Table 1, Table 2 and/or Table 3). In some embodiments, the CTLs recognize two or more T cell epitopes from Table 1, Table 2 and/or Table 3. In some embodiments, the population of CTLs collectively comprise T cell receptors that recognize T cell epitopes from any combination of JCV, BKV, MCV, EBV, CMV, ADV and/or from other viruses. In some embodiments, the population of CTLs collectively comprise T cell receptors that recognize at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 T cell epitopes (e.g., at least 1, 2, 3, 4, 5, 6, or 7 T cell epitopes from Table 1 and/or at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the epitopes listed in Table 1, Table 2 and/or Table 3).

In some aspects, provided herein are methods of presenting or treating a polyomavirus infection (e.g., a BKV, JCV, or MCV infection) or cancer (e.g., a polyomavirus associated cancer, such as a BKV, JVC, or MCV associated cancer) in a subject comprising administering, to a subject, compositions (e.g., therapeutic compositions) comprising the nucleic acid vector described herein, peptides produced by the nucleic acid vector described herein, CTLs and/or APCs provided herein (e.g., comprising the nucleic acid vector described herein) and a pharmaceutically acceptable carrier. In some embodiments, the CTLs and/or APCs are not autologous to the subject. In some embodiments, the T cells and/or APCs are autologous to the subject. In some embodiments, the T cells and/or APCs are stored in a cell bank before they are administered to the subject.

### Pharmaceutical Compositions

In some aspects, provided herein is a composition (e.g., a pharmaceutical composition, such as a vaccine composition), containing a peptide (e.g., comprising an epitope from Table 1), nucleic acid, nucleic acid vector, recombinant adenovirus, antibody, CTL, or an APC described herein formulated together with a pharmaceutically acceptable carrier, as well as methods of treating cancer (e.g., a polyomavirus associated cancer, such as a BKV, JVC, or MCV associated cancer) or a polyomavirus infection (e.g., a BKV, JCV, MCV, CMV, EBV, or ADV infection) using such pharmaceutical compositions. In some embodiments, the composition includes a combination of multiple (e.g., two or more) agents provided herein.

In some embodiments, the pharmaceutical composition further comprises an adjuvant. As used herein, the term “adjuvant” broadly refers to an agent that affects an immunological or physiological response in a patient or subject. For example, an adjuvant might increase the presence of an antigen over time or to an area of interest like a tumor, help absorb an antigen-presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent. Examples of adjuvants include, but are not limited to, an immune modulatory protein, Adjuvant 65, α-GalCer, aluminum phosphate, aluminum hydroxide, calcium phosphate, β-Glucan Peptide, CpG DNA, GPI-0100, lipid A, lipopolysaccharide, Lipovant, Montanide, N-acetyl-muramyl-L-alanyl-D-isoglutamine, Pam3CSK4, quil A and trehalose dimycolate.

Methods of preparing these formulations or compositions include bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions of this invention suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents of the present invention, which may be used in a suitable hydrated form, and/or the pharmaceutical compositions of the present invention, are formulated into pharmaceutically-acceptable dosage forms by conventional methods known to those of skill in the art.

### Therapeutic Methods

In certain aspects, provided herein are methods of treating and/or preventing cancer (e.g., a polyomavirus-associated cancer, such as a BKV-, JCV-, or MCV-associated cancer) or a polyomavirus infection (e.g., a BKV, JCV, or MCV infection). In some embodiments, the method comprises administering to the subject pharmaceutical composition comprising a CTL, APC, polypeptide and/or nucleic acid molecule described herein.

In some embodiments, the subject treated is immunocompromised. For example, in some embodiments, the subject has a T cell deficiency. In some embodiments, the subject has leukemia, lymphoma or multiple myeloma. In some embodiments, the subject is infected with HIV and/or has AIDS. In some embodiments, the subject has undergone a tissue, organ and/or bone marrow transplant. In some embodiments, the subject is being administered immunosuppressive drugs. In some embodiments, the subject has undergone and/or is undergoing chemotherapy. In some embodiments, the subject has undergone and/or is undergoing radiation therapy.

In some embodiments, the subject has cancer. In some embodiments, the methods described herein may be used to treat any cancerous or pre-cancerous tumor. In some embodiments, the cancer expresses one or more of the BKV, MCV or JCV epitopes provided herein (e.g., the BKV or JCV epitopes listed in Tables 1, 2, or 3). In some embodiments, the cancer is Merkel cell carcinoma. In some embodiments, the cancer includes a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous iadenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject is also administered an anti-viral drug that inhibits BKV or JCV replication. For example, in some embodiments, the subject is administered ganciclovir, valganciclovir, foscarnet, cidofovir, acyclovir, formivirsen, maribavir, BAY 38-4766 or GW275175X.

In some embodiments, the subject is also administered an immune checkpoint inhibitor. Immune Checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In some embodiments, a composition provided herein is administered prophylactically to prevent cancer and/or a BKV, MCV or JCV infection. In some embodiments the composition may be administered prior to or after the detection of cancer cells or BKV-, MCV- or JCV-infected cells in a subject. In some embodiments, after administration of a composition comprising peptides, nucleic acids, CTLs, and/or APCs described herein, a proinflammatory response is induced. The proinflammatory immune response comprises production of proinflammatory cytokines and/or chemokines, for example, interferon gamma (IFN-γ) and/or interleukin 2 (IL-2).

Conjunctive therapy includes sequential, simultaneous and separate, and/or co-administration of the active compounds in such a way that the therapeutic effects of the first agent administered have not entirely disappeared when the subsequent treatment is administered. In some embodiments, the second agent may be co-formulated with the first agent or be formulated in a separate pharmaceutical composition.

In some aspects, provided herein is a method of identifying a subject suitable for a therapy provided herein (e.g., methods of treating a BKV, JCV, or MCV infection and/or cancer in a subject comprising administering to the subject a pharmaceutical composition provided herein). In some embodiments, the method comprises isolating a sample from the subject (e.g., a blood sample, a tissue sample, a tumor sample) and detecting the presence of an epitope listed in Tables 1 or 2 in the sample. In some embodiments the epitope is detected using an ELISA assay, a western blot assay, a FACS assay, a fluorescent microscopy assay, an Edman degradation assay and/or a mass spectrometry assay (e.g., protein sequencing). In some embodiments, the presence of the BKV or JCV epitope is detected by detecting a nucleic acid encoding the BKV, MCV or JCV epitope. In some embodiments, the nucleic acid encoding the BKV, MCV or JCV epitope is detected using a nucleic acid probe, a nucleic acid amplification assay and/or a sequencing assay.

In some embodiments, the method comprises HLA typing of the subject. In some embodiments, the subject is identified as suitable for treatment with a method provided herein if the subject expresses an HLA to which an epitope provided herein is restricted. In some embodiments, the methods provided herein further comprise treating the identified subject using a therapeutic method provided herein (e.g., by administering to the subject a pharmaceutical composition provided herein). In some embodiments the subject is administered a composition comprising CTLs described herein, wherein the CTLs comprise TCRs that recognize an epitope provided herein that is HLA restricted to an HLA expressed by the subject. In some embodiments the subject is administered a composition comprising a polypeptide comprising an epitope provided herein that is HLA restricted to an HLA expressed by the subject. In some embodiments the subject is administered a composition comprising an APC presenting a polypeptide comprising an epitope provided herein that is HLA restricted to an HLA expressed by the subject. In some embodiments the subject is administered a composition comprising an nucleic acid encoding a polypeptide comprising an epitope provided herein that is HLA restricted to an HLA expressed by the subject.

### EXAMPLES

### Example 1: CD8⁺ T Cell Responses are Directed Towards LTA and STA, while CD4⁺ T cell Responses are Directed Towards LTA, VP1 and STA

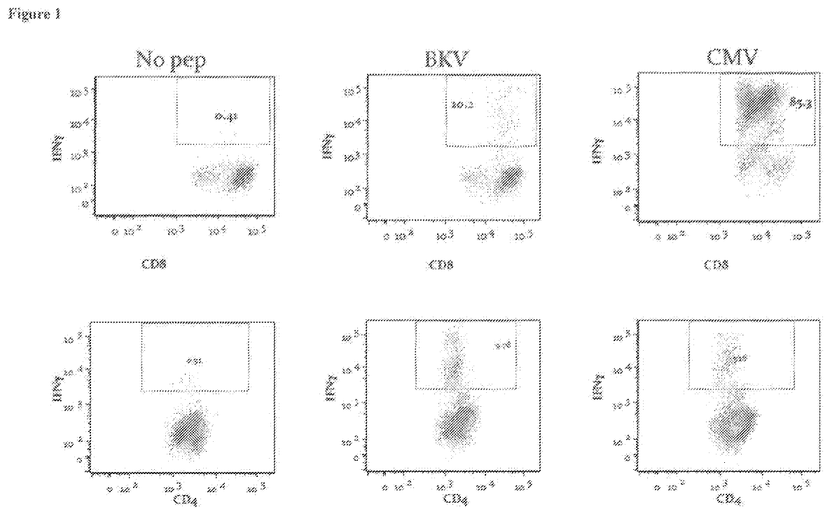

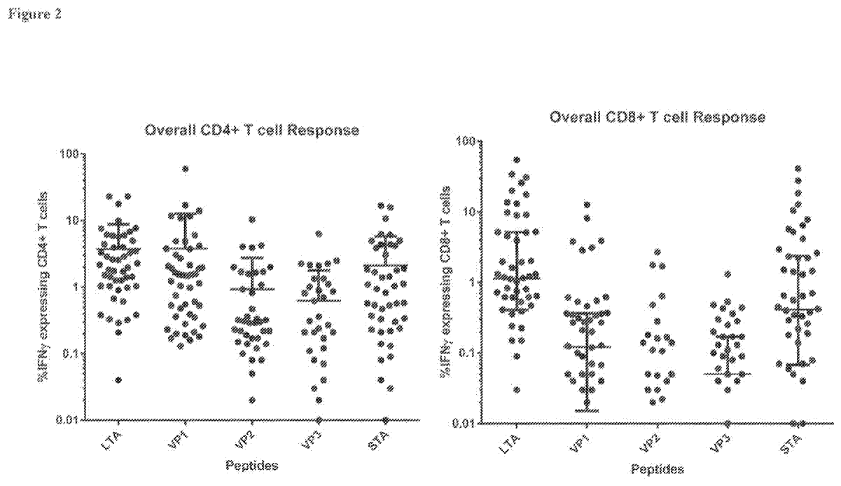

PBMCs from healthy volunteers were incubated with BKV OPPs and cultured these cells for 14 days in the presence of IL-2 and T cell growth factor (TCGF). On day 14, these T cell cultures were assessed for BKV-specificity using ICS assay. FIG. 1 shows that in vitro culture of T cells with BKV peptides for 14 days resulted in expansion of virus-specific T cells. In some cases, these expansions were comparable to CMV-specific T cells. A detailed summary of the T cell assays based on in vitro expanded T cells is presented in FIG. 2. These initial analyses clearly showed that CD8⁺ T cell responses were predominantly directed towards LTA and STA, while CD4⁺ T cell responses were directed towards LTA, VP1 and STA. To validate these observations, T cell assays were repeated in 50 volunteers (including many volunteers from the first set of assays) and a summary of this analysis are presented in FIG. 2. Consistent with the data presented in FIG. 2, dominant CD8⁺ and CD4⁺ T cell responses were detected towards LTA, STA and VP1 antigens.

### Example 2: Further Characterization of T Cell Responses

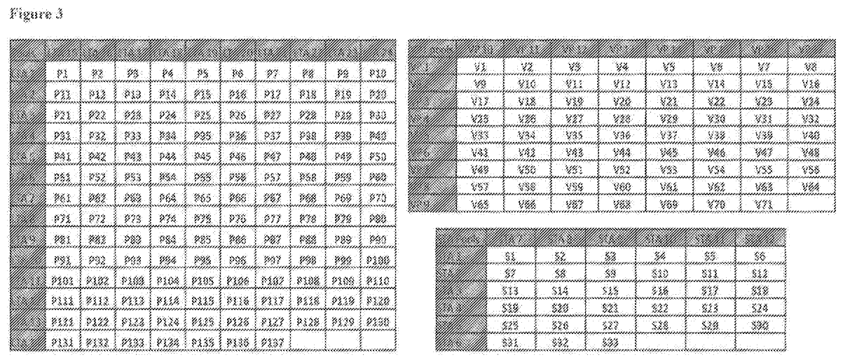

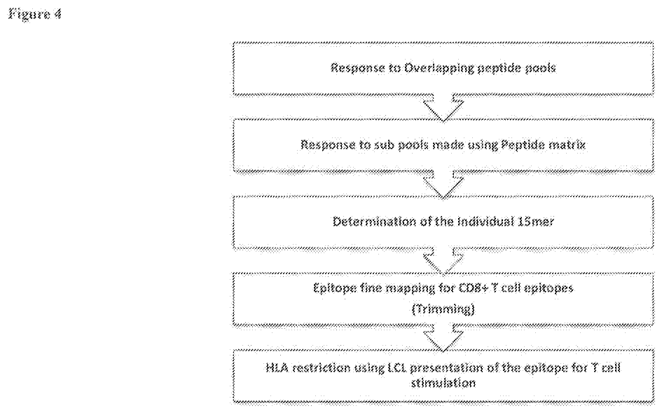

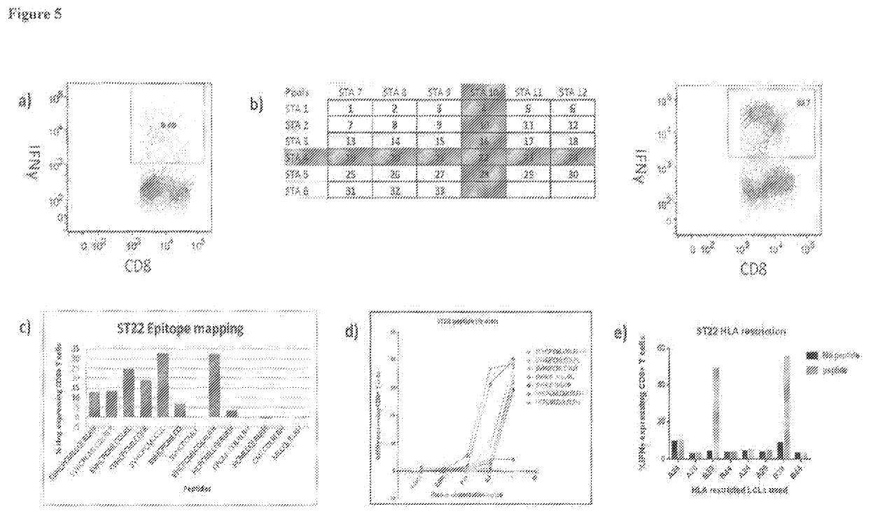

In order to characterize the T cell responses directed towards these antigens and precisely map the HLA class I and class II-restricted T cell responses, individual overlapping peptides (15 aa long overlapping by 10 aa) were sourced for LTA, STA and VP1 proteins for T cell epitope mapping. A two-dimensional peptide matrix was used to distribute all individual peptides into small overlapping peptide pools. The matrix is set up in the way that each peptide occurs once on the ordinate (FIG. 3). These peptide pools were used in ICS assays. After the ICS analysis, T cell response to the peptide pools was compared with the matrix to identify individual peptides. These individual peptides were further assessed for T cell expansion and ICS analysis to identify potential BK. Once the 15 mer peptide was identified, further mimimalization of the epitope sequence was carried out to identify the optimal T cell epitope sequence. The 15 mer peptide sequences were trimmed from both N- and C-terminus to a minimal of 9 aa long peptides. Once the minimal peptide sequence was identified, further confirmation was carried out using limiting dose titration ICS assay. After mapping minimal epitope sequence, the HLA restriction of the epitope was identified by stimulating T cells using peptide loaded HLA-matched and mismatched LCLs. The complete process of epitope mapping is shown in the flowchart provided in FIG. 4.

Representative data from one of the BKV epitope mapping process is shown in FIG. 5. Data presented in FIG. 5, Panel A shows that BKV-specific T cells from healthy volunteer H26 recognized STA OPP. In order to map the T cell epitope further analysis was carried out using sub pools of STA peptides (12 pools) designed based on the two dimensional matrix shown in FIG. 3. Intracellular cytokine analysis based on STA peptides showed that pools 4 and 10 were efficiently recognized by CD8⁺ T cells, which when overlayed on to the matrix layout showed STA22 peptide as the common peptide sequence among the responding pools (FIG. 5, Panel B). The peptide trimming process showed VHCPCMLCQL (SEQ ID NO: 8) to be the T cell epitope (FIG. 5, Panel C and D). The HLA restriction analysis using the HLA matched LCLs showed VHCPCMLCQL (SEQ ID NO: 8) to be an HLA B*39-restricted epitope (FIG. 5, Panel E). Similar epitope mapping process was carried out for other CD4⁺ and CD8⁺ T cell epitopes. The list of CD8⁺ and CD4⁺ BKV epitopes mapped during this study is listed in Table 4 and 5, respectively.

TABLE 4
CD8+ epitopes
CD8 Epitopes

| | | HLA | SEQ |
|---|---|---|---|
| Epitope | Antigen | Restriction | ID NO.: |
| DSQHSTPPK | LTA | A*11 | 1 |
| AVDTVLAKK | LTA | A*11 | 2 |
| CYCIDCFTQ | STA | A*24 | 3 |
| LPLMRKAYL | LTA/STA | B*07/B*08 | 4 |
| FPLCPDTLY | STA | B*35 | 5 |
| TLYCKEWPI | STA | B*35 | 6 |
| EPLVWIDCY | STA | B*35 | 158 |
| VHCPCMLCQL | STA | B*39 | 8 |
| NREESMELMDL | LTA/STA | B*40 | 9 |
| MELMDLLGL | LTA/STA | B*40 | 10 |
| FFAVGGDPLEM | STA | B*40 | 11 |
| YCIDCFTQW | STA | B*57 | 159 |
| TPHRHRVSA | LTA | B*56 | 13 |
| LLLGMYLEF | LTA | A*29 | 14 |
| VFLLLGMYLEF | LTA | A*23 | 160 |
| IEESIQGGL | LTA | B*40 | 161 |
| TEVIGITSML | VP1 | B*40 | 162 |
| ARIPLPNL | VP1 | B*27 | 18 |
| VKNPYPISFLL | VP1 | Cw*07 | 19 |
| QAVDTVLAKK | LTA | A*11 | 20 |
| MLTERFNHIL | LTA | A*02 | 163 |
| LLLIWFRPV | LTA | A*02:01 | 22 |
| AITEVECFL | VP1 | A*02:01 | 164 |
| RLDSEISMY | LTA | A*01 | 165 |
| SVKVNLEKK | LTA | A*03 | 166 |
| AYLRKCKEF | LTA | A*24 | 167 |
| LPGDPDMIRYIDRQG | VP1 | A24/A29/B7/B39 | 28 |
| LEVKTGVDAITEVEC | VP1 | A24/A29/B7/B39 | 29 |
| DICGLFTNSSGTQQW | VP1 | A 24/A29/B7/B39 | 168 |
| ESQVEEVRVFDGTEQ | VP1 | A24/A29/B7/B39 | 31 |

TABLE 5
CD4+ epitopes
CD4 Epitopes

| | | HLA | SEQ |
|---|---|---|---|
| Epitope | Antigen | Restriction | ID NO.: |
| GTQQWRGLARYFKIR | VP1 | DRB1*11/8 | 32 |
| RGLARYFKIRLRKRS | VP1 | DRB1*11 | 33 |
| RKAYLRKCKEFHPDK | LTA | DRB1*13 | 34 |
| WDEDLFCHEDMFASD | LTA | DQB5*01 | 35 |
| CFTQWFGLDLTEETL | STA | DRB1*03/04 | 36 |
| GGDEDKMKRMNTLYK | LTA/STA | DRB1*13 | 37 |
| KMKRMNTLYKKMEQD | LTA/STA | DRB1*13 | 38 |
| FNVPKRRYWLFKGPI | LTA | DRB1*15 | 39 |
| RRYWLFKGPIDSGKT | LTA | DRB1*15 | 40 |
| VGPLCKADSLYVSAA | VP1 | ND* | 41 |
| AYLDKNNAYPVECWI | VP1 | ND* | 42 |
| DMIRYIDRQGQLQTK | VP1 | ND* | 43 |

### Example 3: Profiling Functional and Phenotypic Characteristics of BKV Specific T Cells in Healthy Individuals and Transplant Recipients

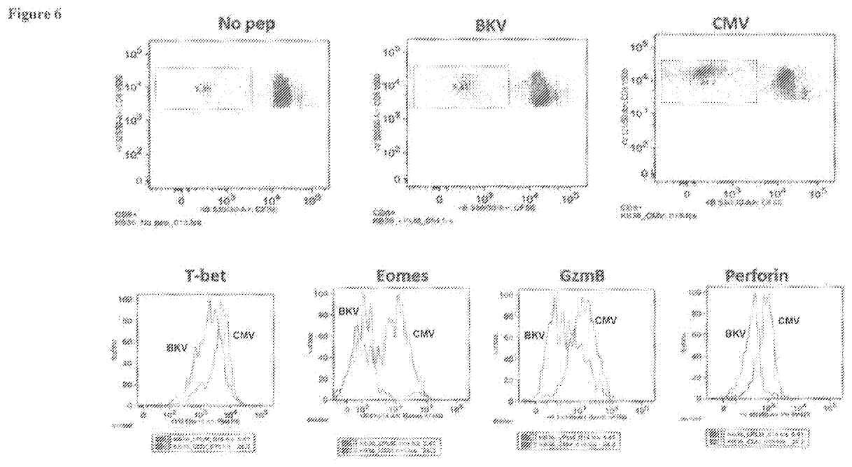

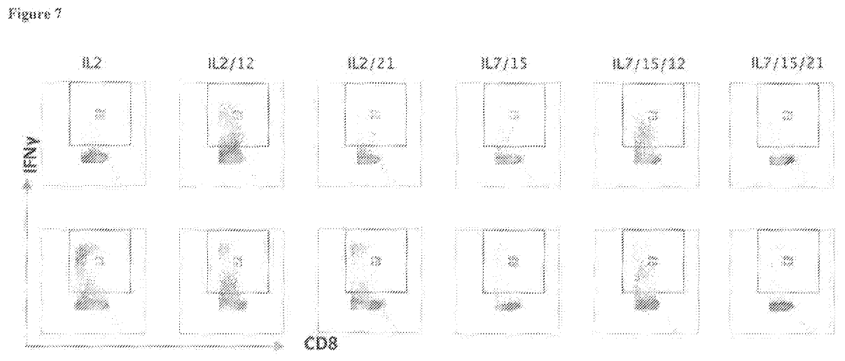

In recent years, the T-box transcription factors (T-bet) and Eomesodermin (Eomes) have been shown to play important roles in determining the fate of CD8+ T cells during infection. High levels of T-bet are associated with the cytotoxic T cell differentiation and upregulation of perforin and Granzyme B in antigen specific cells. A high level of Eomes is associated with the long term memory formation. It has been seen in various studies that their cooperative expression is critical for infection control. In mouse studies it has also been shown that the deletions of either of the transcription factors have resulted in failure to control infection. Hence it is critical to study the expression of these transcription factors which could help in the understanding the phenotypic characterization of the T cells and T cell differentiation during both acute and chronic viral infections. The expression patterns of T-bet and Eomes in BKV specific T cells is not yet been understood, and the analysis of the transcription factors on these T cells may enable a deeper understanding on the differentiation of BKV specific T cells. A detailed study on the functional characteristics of T cells could also lead to development of effective immunotherapy for BKV associated diseases. An initial set of experiments have started to study the transcriptional factors on the T cells which regulate the differentiation of the T cells. The expression of T-bet, Eomes, perforin and granzyme B were assayed on the BKV specific T cells and CMV specific T cells using ICS. The initial analysis showed a medium to low level of T bet expression in BKV specific T cells while high levels of T-bet was seen with CMV specific T cells (FIG. 6). Also, very low expression of Eomes was found with BKV specific T cells in comparison to the CMV specific T cells. Low levels of perforin and granzyme B was also seen with BKV specific T cells. This preliminary data suggests that BKV specific T cells could be functionally low in effector function. Hence driving the effector function of BKV specific CTLs will be the focus of my study which could help in developing an effective adoptive T cell immunotherapy.

### Example 4: BKV-specific T cell Expansion

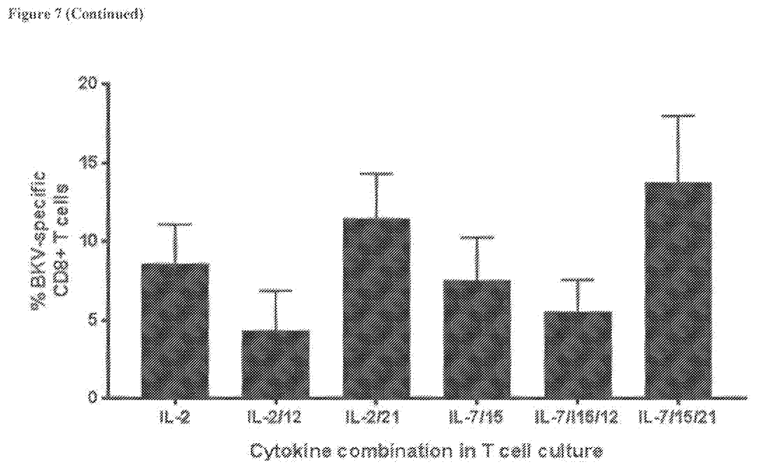

BKV-specific T cells were expanded in vitro following stimulation with pooled BKV epitopes. Specifically, PBMC from healthy volunteers were stimulated with synthetic BKV peptides (Table 1) for 1 hour and then cultured for 12-14 days in the presence different cytokine combinations, including IL-2 (10 ng/ml), IL-21 (30 ng/ml), IL7 (10 ng/ml), IL12 (10 ng/ml) and/or IL15 (10 ng/ml). The BKV specificity of the expanded T cells was assessed using standard intracellular cytokine assays (FIG. 7).

### Example 5: Generation of Consensus Alignments

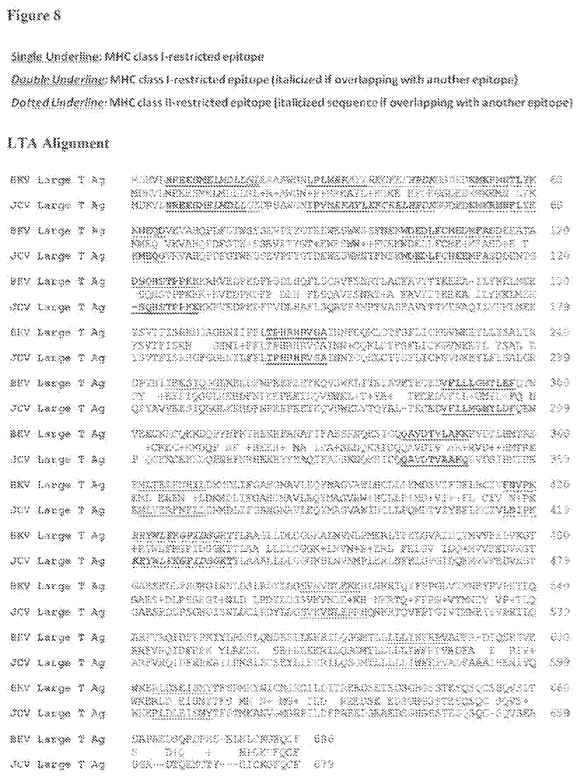

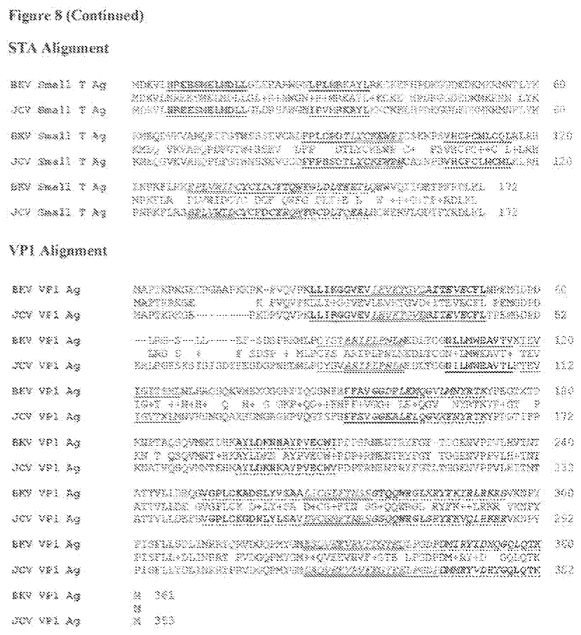

To identify JC virus epitopes homologous to BKV epitopes described herein, the NCBI Blastp sequence alignment program was used to align the amino acid sequences of the BKV and JCV Large T Antigen (LTA) protein, Small T Antigen protein (STA), and VP1 protein, respectively, and homologous sequences were identified (FIG. 8, epitopes highlighted). The NCBI Blastp sequence alignment program was also used to align the amino acid sequences of the BKV and MCV VP1 protein to identify homologous sequences (FIG. 9).

### Example 6:Expansion of CTLs in the Presence of IL-21

BKV specific T cells were generated using the PBMCs from healthy donors. PBMCs were stimulated in vitro with respective BKV peptide pools at a concentration of 1 μg/ml and incubated at 37° C., 6.5% CO₂ for an hour. The cells were then washed and split into two to be cultured in two conditions. A part of the cells was grown in the R10 medium (RPMI+10% FCS) containing 30 ng/ml of IL21 (Milteyni Biotech Ltd) in 24 well plates incubated at 37° C., 6.5% CO₂. Another part of the cells was incubated with R10 medium without IL-21. The cultures grown in both conditions were supplemented with R10 medium containing recombinant interleukin-2 (Charles River Laboratory, NIH, USA) at 20 IU/ml on day 2 and then supplemented with media containing IL-2 every 3 days thereafter until day 20.

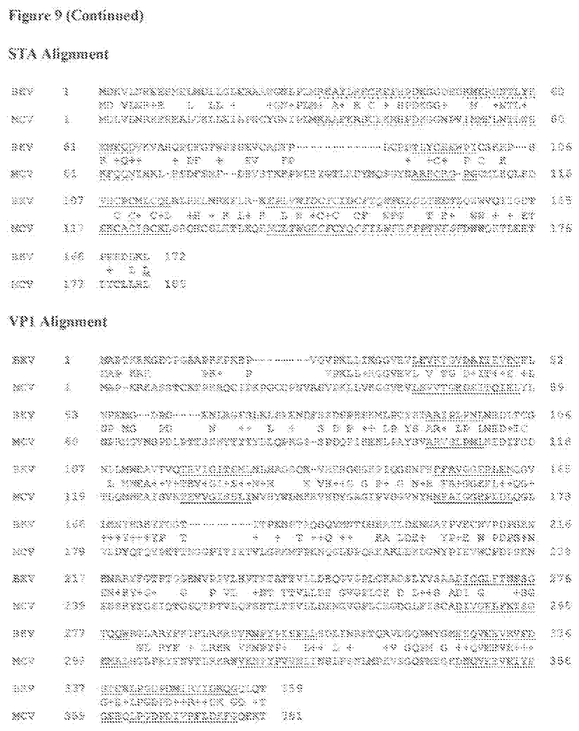

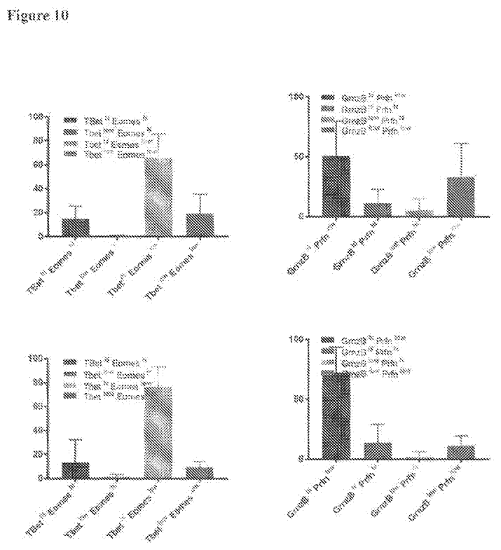

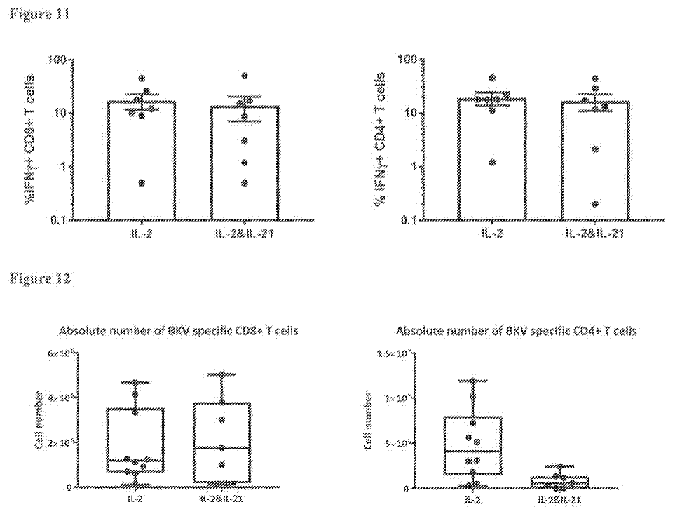

T cells in the cultures were counted and required amount of cells were used for an IFN-γ intracellular cytokine (ICS) assay while the remaining cells were cryopreserved in liquid nitrogen. Approximately 2×10⁵ of CTLs were added to a 96 well V-bottom plate. Cells were stimulated with respective peptides at a concentration of 1 μg /ml in R10 medium containing Golgiplug Brefeldin A (BD Pharmingen, San Diego, Calif.) and incubated at 37° C., 6.5% CO₂ for four hours. BKV specific T cells were recalled with both BKV peptide and its respective JCV variant and vice versa for JCV specific T cells. After incubation, the cells were washed with PBS containing 2% FBS (wash buffer) and the pellet was resuspended in 50 μL of wash buffer containing FITC-conjugated anti-CD4 and PerCP-Cy5.5 conjugated anti-CD8 antibodies and incubated at 4° C. for 30 minutes. Cells were then washed twice with PBS, fixed and permeabilized with Cytofix/Cytoperm solution (BD Pharmingen) for 20 mins. Cells were then washed and incubated with PE- anti-IFN-γ antibody diluted in Permwash buffer at 4° C. for 30 minutes. Stained cells were washed twice with Permwash buffer, resuspended in PBS containing 1% paraformaldehyde and acquired using using a BD LSR Fortessa. Post-acquisition analysis was conducted using FlowJo software (TreeStar). IFN-γ expression of the cell populations is provided in FIG. 11, while the numbers of CD4 and CD8 cells in the expanded cultures is provided in FIG. 12.

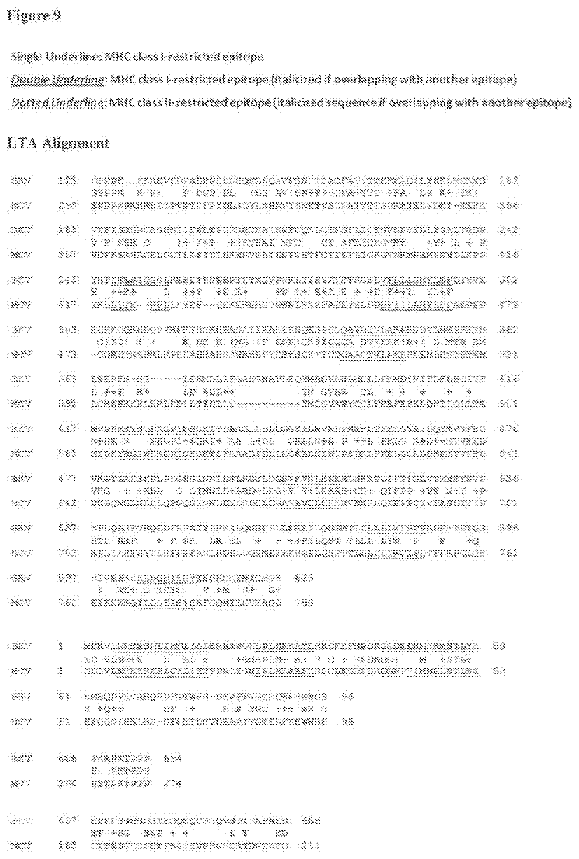

The effect of the presence of IL-21 in the culture on transcription factor and effector molecule expression was tested. Approximately 2×10⁵ of CTLs grown in both conditions were added to a 96 well V-bottom plate. The cells were washed with PBS containing 2% FBS (wash buffer) and the pellet was resuspended in 50 μL of PBS containing 1 μl of respective APC conjugated BKV specific dextramer and incubated at 4° C. for 20 minutes. Cells were then added with PE-Cy7 CD4 and V500 CD8 antibodies and incubated at 4° C. for 30 minutes. After incubation, cells were washed twice with PBS, fixed and permeabilized with transcription factor Cytofix/Cytoperm solution (BD Pharmingen) for 1 hour. Cells were then washed and incubated with eflour710-anti-Eomes antibody, AF100-conjugated-anti-GranzymeB, BV421 conjugated anti-Perforin and PE conjugated anti-Tbet antibodies diluted in Permwash buffer at 4° C. for 30 minutes. Stained cells were washed twice with Permwash buffer, resuspended in PBS containing 1% paraformaldehyde and acquired using using a BD LSR Fortessa. Post-acquisition analysis was conducted using FlowJo software (TreeStar). Transcription factor and effector molecule expression in the cell populations shown in FIG. 10.

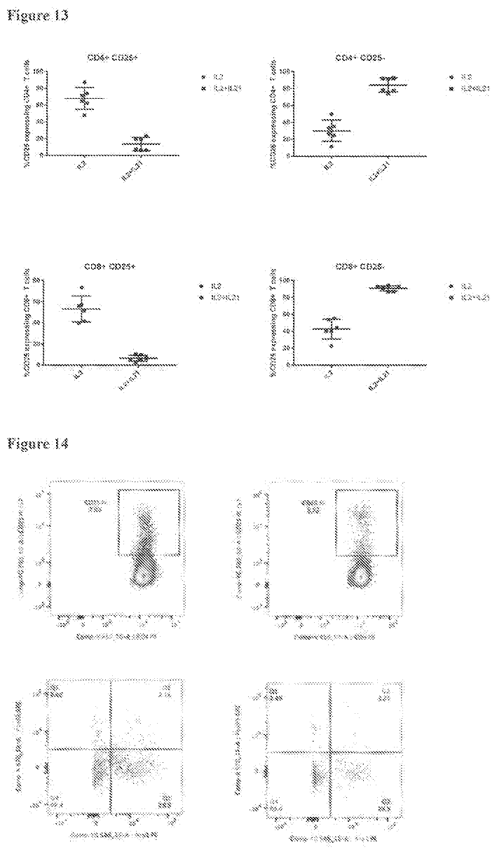

The effect of the presence of IL-21 on expansion of regulatory T cells was also tested. Approximately 2×10⁵ of CTLs grown in both conditions were added to a 96 well V-bottom plate. The cells were washed with PBS containing 2% FBS (wash buffer) and the pellet was resuspended in 50 μL of PBS containing FITC conjugated anti-CD3, Pacific blue conjugated anti-CD4, PEcy7 conjugated anti-CD25, PE-conjugated anti-neuropilin1, and BV786 conjugated anti-CD127 antibodies and incubated at 4° C. for 30 minutes. Cells were then mixed with PE-Cy7 CD4 and V500 CD8 antibodies and incubated at 4° C. for 30 minutes. After incubation, Cells were washed twice with PBS, fixed and permeabilized with FoxP3 Cytofix/Cytoperm solution (ebiosciences Ltd) for 1 hour. Cells were then washed and incubated with APC conjugated anti-FoxP3 antibody diluted in Permwash buffer at 4° C. for 30 minutes. Stained cells were washed twice with Permwash buffer, resuspended in PBS containing 1% paraformaldehyde and acquired using a BD LSR Fortessa. Post-acquisition analysis was conducted using FlowJo software (TreeStar). The presence of regulatory T cells in the cell populations is shown in FIGS. 13 and 14.

### Example 7: T cell Cross-Reactivity

JCV variants for mapped BKV epitopes were synthesized. BKV and JCV specific T cells were generated using the PBMCs from healthy donors. PBMCs were washed and resuspended in R10 (RPMI+10% FCS). The cells were then stimulated in vitro with respective BKV and JCV peptide separately at a concentration of 1 μg/ml and incubated at 37° C., 6.5% CO₂ for an hour. The cells were then washed and grown for 14 days in 24 well plates incubated at 37° C., 6.5% CO₂. The cultures were supplemented with R10 medium containing recombinant interleukin-2 (Charles River Laboratory, NIH, USA) at 20 IU/ml on day 2 and then supplemented with R10 medium containing IL-2 every three days thereafter until day 14. On day 14, T cells in the cultures were counted using the Trypan Blue exclusion method and required amount of cells were used for an IFN-γ intracellular cytokine (ICS) assay while the remaining cells were cryopreserved in liquid nitrogen.

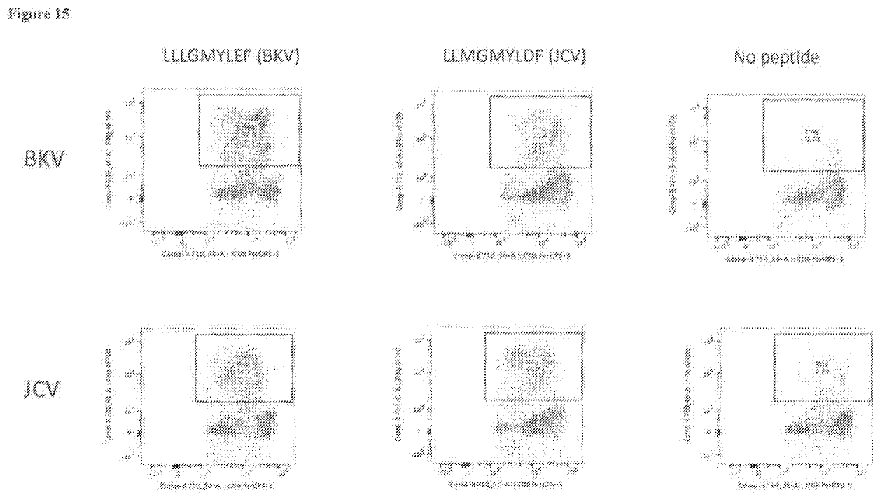

T cell cross reactivity was determined by measuring IFN-γ expression following T cell restimulation with BKV or JCV epitopes. Approximately 2×10⁵ of CTLs were added to a 96 well V-bottom plate. Cells were stimulated with respective peptides at a concentration of 1 μg/ml in R10 medium containing Golgiplug Brefeldin A (BD Pharmingen, San Diego, Calif.) and incubated at 37° C., 6.5% CO₂ for four hours. BKV specific T cells were recalled with both BKV peptide and its respective JCV variant and vice versa for JCV specific T cells. After incubation, the cells were washed with PBS containing 2% FBS (wash buffer) and the pellet was resuspended in 50 μL of wash buffer containing FITC-conjugated anti-CD4 and PerCP-Cy5.5 conjugated anti-CD8 antibodies and incubated at 4° C. for 30 minutes. Cells were then washed twice with PBS, fixed and permeabilized with Cytofix/Cytoperm solution (BD Pharmingen) for 20 mins. Cells were then washed and incubated with PE- anti-IFN-γ antibody diluted in Permwash buffer at 4° C. for 30 minutes. Stained cells were washed twice with Permwash buffer, resuspended in PBS containing 1% paraformaldehyde and acquired using using a BD LSR Fortessa. Post-acquisition analysis was conducted using FlowJo software (TreeStar). Representative IFN-γ expression data is shown in FIG. 15.

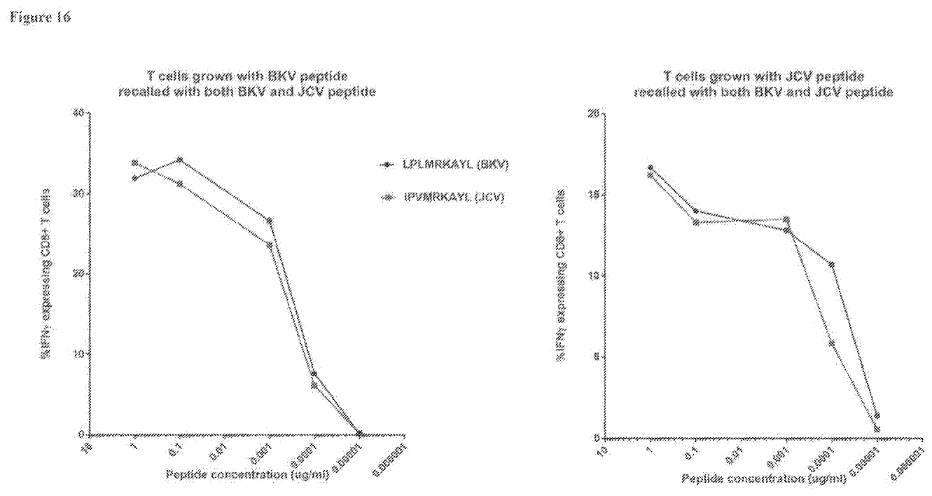

The peptides that responded both in BKV and JCV specific T cells were further analysed for avidity using limiting dose titration assay. The peptides were titrated 10 fold starting from 1 μg/ml up to a concentration of 10⁻⁵ μg/ml. These titrated peptides were then used to recall the BKV and JCV specific CTLs in standard IFN-γ intracellular cytokine assay. Representative titration assay data is shown in FIG. 16.

Epitope cross-reactivity is provided in Table 6 (for CD8 epitopes) and Table 7 (for CD4 epitopes).

TABLE 6
BKV/JCV Cross-reactivity of
exemplary CD8 epitopes.
CD8 epitopes

| | | | | BKV and JCV |
|---|---|---|---|---|
| | SEQ | | SEQ | Cross- |
| BKV sequence | ID NO: | JCV sequence | ID NO: | reactivity |
| NREESMELMDL | 9 | NREESMELMDL | 170 | Yes |
| MELMDLLGL | 10 | MELMDLLGL | 53 | Yes |
| SQHSTPPKK | 121 | SQHSTPPKK | 124 | Yes |
| TPHRHRVSA | 13 | TPHRHRVSA | 56 | Yes |
| YFLLLGMYLEF | 160 | VFLLMGMYLDF | 58 | Yes |
| AVDTVLAKK | 2 | AVDTVAAKQ | 45 | No |
| FPLCPDTLYC | 122 | FPPNSDTLYC | 125 | No |
| VHCPCMLCQL | 8 | VHCPCLMCML | 51 | Yes |
| EPLVWIDCY | 158 | SPLVWIDCY | 171 | Yes |
| CYCIDCFTQ | 3 | CYCFDCFRQ | 46 | No |
| YCIDCDTQW | 159 | YCFDCFWQW | 55 | No |
| LLIKGGVEV | 123 | LLIRGGVEV | 172 | Yes |
| AITEVECFL | 164 | SITEVECFL | 173 | Yes |
| NLLMWEAVTV | 169 | NILMWEAVTL | 174 | No |
| FFAVGGDPLEM | 11 | FFSVGGEALEL | 54 | No |
| LLLGMYLEF | 14 | LLMGMYLDF | 57 | Yes |
| LPLMRKAYL | 4 | IPVMRKAYL | 47 | Yes |

TABLE 7
BKV/JCV Cross reactivity of exemplary CD4 epitopes
CD4 epitopes

| | | | | BKV and JCV |
|---|---|---|---|---|
| BKV sequence | SEQ ID NO: | JCV sequence | SEQ ID NO: | Cross-reactivity |
| DMIRYIDRQGQLQTK | 43 | DMMRYVDRYGQLQTK | 86 | No |
| GTQQWRGLARYFKIR | 32 | GSQQWRGLSRYFKVQ | 75 | Yes |
| RGLARYFKIRLRKRS | 33 | RGLSRYFKVQLRKRR | 76 | No |
| WDEDLFCHEDMFASD | 35 | WDEDLFCHEEMFASD | 78 | No |
| RKAYLRKCKEFHPDK | 34 | RKAYLKKCKELHPDK | 77 | No |
| GGDEDKMKRMNTLYK | 37 | GGDEDKMKRMNFLYK | 80 | No |
| KMKRMNTLYKKMEQD | 38 | KMKRMNFLYKKMEQG | 81 | No |
| RNVPKRRYWLFKGPI | 39 | KNIPKKRYWLFKGPI | 175 | Yes |
| RRYWLFKGPIDSGKT | 40 | KRYWLFKGPIDSGKT | 83 | No |
| CFTQWFGLDLTEETL | 36 | CFRQWFGCDLTQEAL | 79 | No |
| AYLDKNNAYPVECWI | 42 | AYLDKNKAYPVECWV | 85 | No |
| VGPLCKADSLYVSAA | 41 | VGPLCKGDNLYLSAV | 84 | No |

1. An isolated protein comprising one or more of the epitopes listed in Tables 1-3.

2. The isolated protein of claim 1, wherein the one or more epitopes comprises a BK virus (BKV) epitope listed in Table 1, a JC virus (JCV) epitope listed in Table 2, or a hybrid epitope according to Table 3.

3-4. (canceled)

5. The isolated protein of claim 1, wherein the peptide comprises a plurality of epitopes listed in Tables 1-3.

6-8. (canceled)

9. The isolated protein of claim 5, further comprising an intervening amino acid sequence between at least two of the plurality of epitopes.

10-11. (canceled)

12. The isolated protein of claim 1, wherein the epitopes have HLA class I restrictions to HLA-A1, -A2, -A3, -A11, -A23, -A24, -A26, -A29, -A30, -B7, -B8, -B27, -B35, -B38, -B40, -B41, -B44, -B51, -B56, -B57 or -B58.

13. The isolated protein of claim 1, wherein the epitopes have HLA class II restrictions to HLA-DP, -DM, -DOA, -DOB, -DQ, or -DR.

14. (canceled)

15. The isolated protein of claim 1, wherein the isolated protein comprises epitope amino acid sequences set forth in SEQ ID NOS: 5, 6, 36, 41 and 42.

16-17. (canceled)

18. The isolated protein of claim 1, wherein the isolated protein further comprises one or more epitopes from Merkel cell virus (MCV).

19. The isolated protein of claim 1, further comprising one or more epitopes from a non-polyomavirus.

20. (canceled)

21. An isolated nucleic acid encoding the isolated protein of claim 1.

22. An expression construct comprising the isolated nucleic acid of claim 21.

23-24. (canceled)

25. A pharmaceutical composition comprising the isolated protein of claim 1 and a pharmaceutically acceptable carrier.

26. (canceled)

27. A vaccine composition comprising the isolated protein of claim 1 and a pharmaceutically acceptable carrier.

28-52. (canceled)

53. A method of treating or preventing a cancer in a subject, the method comprising administering to the subject a pharmaceutical composition comprising cytotoxic T cells (CTLs) comprising T cell receptors (TCRs) that recognize one or more epitopes listed in Tables 1-3.

54-56. (canceled)

57. The method of claim 53, wherein the cancer is a BK virus (BKV), a JC virus (JCV), or a Merkel Cell Virus (MCV) associated cancer.

58-60. (canceled)

61. A method of treating or preventing a polyomavirus infection in a subject, the method comprising administering to the subject a pharmaceutical composition comprising cytotoxic T cells (CTLs) comprising T cell receptors (TCRs) that recognize one or more epitopes listed in Tables 1-3.

62-64. (canceled)

65. The method of claim 61, wherein the polyomavirus is a BK virus (BKV), a JC virus (JCV), or a Merkel Cell Virus (MCV).

66-67. (canceled)

68. The method of claim 53, wherein the TCRs recognize an epitope shared by two or more polyomaviruses.

69. The method of claim 68, wherein the shared epitope comprises a region of sequence homology between the at least two polyomaviruses, and the region of sequence homology is at least three amino acids across the full length of the epitope sequence.

70-93. (canceled)

93. A method of inducing proliferation of polyomavirus-specific cytotoxic T cells (CTLs), comprising contacting CTLs with antigen-presenting cells (APCs) that present a polyomavirus peptide comprising one or more epitopes listed in Tables 1-3.

94. The method of claim 93, wherein the one or more epitopes comprise a BK virus (BKV) epitope listed in Table 1.

95. The method of claim 93, wherein the one or more epitopes comprise a JC virus (JCV) epitope listed in Table 2.

96. The method of claim 93, wherein the one or more epitopes comprise a hybrid epitope according to Table 3.

97-141. (canceled)


SEQUENCE LISTING (175 sequences)

SEQ ID NO: 1 - protein, 9 aa, BK polyomavirus
DSQHSTPPK

SEQ ID NO: 2 - protein, 9 aa, BK polyomavirus
AVDTVLAKK

SEQ ID NO: 3 - protein, 9 aa, BK polyomavirus
CYCIDCFTQ

SEQ ID NO: 4 - protein, 9 aa, BK polyomavirus
LPLMRKAYL

SEQ ID NO: 5 - protein, 9 aa, BK polyomavirus
FPLCPDTLY

SEQ ID NO: 6 - protein, 9 aa, BK polyomavirus
TLYCKEWPI

SEQ ID NO: 7 - protein, 9 aa, BK polyomavirus
  MOD_RES (4)..(4): V or G
  MOD_RES (6)..(6): K or I
EPLXWXDCY

SEQ ID NO: 8 - protein, 10 aa, BK polyomavirus
VHCPCMLCQL

SEQ ID NO: 9 - protein, 11 aa, BK polyomavirus
NREESMELMDL

SEQ ID NO: 10 - protein, 9 aa, BK polyomavirus
MELMDLLGL

SEQ ID NO: 11 - protein, 11 aa, BK polyomavirus
FFAVGGDPLEM

SEQ ID NO: 12 - protein, 9 aa, BK polyomavirus
  MOD_RES (8)..(8): Q or E
YCIDCFTXW

SEQ ID NO: 13 - protein, 9 aa, BK polyomavirus
TPHRHRVSA

SEQ ID NO: 14 - protein, 9 aa, BK polyomavirus
LLLGMYLEF

SEQ ID NO: 15 - protein, 11 aa, BK polyomavirus
  MOD_RES (2)..(2): F or L
VXLLLGMYLEF

SEQ ID NO: 16 - protein, 9 aa, BK polyomavirus
  MOD_RES (6)..(6): Q or H
IEESIXGGL

SEQ ID NO: 17 - protein, 10 aa, BK polyomavirus
  MOD_RES (4)..(4): I or M
TEVXGITSML

SEQ ID NO: 18 - protein, 8 aa, BK polyomavirus
ARIPLPNL

SEQ ID NO: 19 - protein, 11 aa, BK polyomavirus
VKNPYPISFLL

SEQ ID NO: 20 - protein, 10 aa, BK polyomavirus
QAVDTVLAKK

SEQ ID NO: 21 - protein, 10 aa, BK polyomavirus
  MOD_RES (4)..(4): E or D
MLTXRFNHIL

SEQ ID NO: 22 - protein, 9 aa, BK polyomavirus
LLLIWFRPV

SEQ ID NO: 23 - protein, 9 aa, BK polyomavirus
  MOD_RES (4)..(4): E or Q
AITXVECFL

SEQ ID NO: 24 - protein, 9 aa, BK polyomavirus
  MOD_RES (1)..(1): R or K
XLDSEISMY

SEQ ID NO: 25 - protein, 9 aa, BK polyomavirus
SVKVNLEKH

SEQ ID NO: 26 - protein, 9 aa, BK polyomavirus
  MOD_RES (5)..(5): K or R
AYLRXCKEF

SEQ ID NO: 27 - protein, 10 aa, BK polyomavirus
  MISC_FEATURE (1)..(1): May or may not be present
NILMWEAVTL

SEQ ID NO: 28 - protein, 15 aa, BK polyomavirus
LPGDPDMIRYIDRQG

SEQ ID NO: 29 - protein, 15 aa, BK polyomavirus
LEVKTGVDAITEVEC

SEQ ID NO: 30 - protein, 15 aa, BK polyomavirus
  MOD_RES (7)..(7): T or I
DICGLFXNSSGTQQW

SEQ ID NO: 31 - protein, 15 aa, BK polyomavirus
ESQVEEVRVFDGTEQ

SEQ ID NO: 32 - protein, 15 aa, BK polyomavirus
GTQQWRGLARYFKIR

SEQ ID NO: 33 - protein, 15 aa, BK polyomavirus
RGLARYFKIRLRKRS

SEQ ID NO: 34 - protein, 15 aa, BK polyomavirus
RKAYLRKCKEFHPDK

SEQ ID NO: 35 - protein, 15 aa, BK polyomavirus
WDEDLFCHEDMFASD

SEQ ID NO: 36 - protein, 15 aa, BK polyomavirus
CFTQWFGLDLTEETL

SEQ ID NO: 37 - protein, 15 aa, BK polyomavirus
GGDEDKMKRMNTLYK

SEQ ID NO: 38 - protein, 15 aa, BK polyomavirus
KMKRMNTLYKKMEQD

SEQ ID NO: 39 - protein, 15 aa, BK polyomavirus
FNVPKRRYWLFKGPI

SEQ ID NO: 40 - protein, 15 aa, BK polyomavirus
RRYWLFKGPIDSGKT

SEQ ID NO: 41 - protein, 15 aa, BK polyomavirus
VGPLCKADSLYVSAA

SEQ ID NO: 42 - protein, 15 aa, BK polyomavirus
AYLDKNNAYPVECWI

SEQ ID NO: 43 - protein, 15 aa, BK polyomavirus
DMIRYIDRQGQLQTK

SEQ ID NO: 44 - protein, 10 aa, John Cunningham polyomavirus
  MOD_RES (3)..(3): Q or R
  MOD_RES (9)..(9): K or R
KSXHSTPPXK

SEQ ID NO: 45 - protein, 9 aa, John Cunningham polyomavirus
AVDTVAAKQ

SEQ ID NO: 46 - protein, 9 aa, John Cunningham polyomavirus
CYCFDCFRQ

SEQ ID NO: 47 - protein, 9 aa, John Cunningham polyomavirus
IPVMRKAYL

SEQ ID NO: 48 - protein, 9 aa, John Cunningham polyomavirus
FPPNSDTLY

SEQ ID NO: 49 - protein, 9 aa, John Cunningham polyomavirus
FLYCKEWPN

SEQ ID NO: 50 - protein, 9 aa, John Cunningham polyomavirus
  MOD_RES (5)..(5): W or R
SPLVXIDCY

SEQ ID NO: 51 - protein, 10 aa, John Cunningham polyomavirus
VHCPCLMCML

SEQ ID NO: 52 - protein, 12 aa, John Cunningham polyomavirus
NREESMELMDLL

SEQ ID NO: 53 - protein, 9 aa, John Cunningham polyomavirus
MELMDLLGL

SEQ ID NO: 54 - protein, 11 aa, John Cunningham polyomavirus
FFSVGGEALEL

SEQ ID NO: 55 - protein, 9 aa, John Cunningham polyomavirus
YCFDCFRQW

SEQ ID NO: 56 - protein, 9 aa, John Cunningham polyomavirus
TPHRHRVSA

SEQ ID NO: 57 - protein, 9 aa, John Cunningham polyomavirus
LLMGMYLDF

SEQ ID NO: 58 - protein, 11 aa, John Cunningham polyomavirus
VFLLMGMYLDF

SEQ ID NO: 59 - protein, 9 aa, John Cunningham polyomavirus
  MOD_RES (3)..(3): E or G
VEXSIQGGL

SEQ ID NO: 60 - protein, 10 aa, John Cunningham polyomavirus
  MOD_RES (4)..(4): I or L
TEVXGVTLMN

SEQ ID NO: 61 - protein, 9 aa, John Cunningham polyomavirus
ARIPLPNLN

SEQ ID NO: 62 - protein, 11 aa, John Cunningham polyomavirus
VKNPYPISFLL

SEQ ID NO: 63 - protein, 15 aa, John Cunningham polyomavirus
LPGDPDMMRYVDKYG

SEQ ID NO: 64 - protein, 15 aa, John Cunningham polyomavirus
LEVKTGVDSITEVEC

SEQ ID NO: 65 - protein, 15 aa, John Cunningham polyomavirus
DVCGMFTNRSGSQQW

SEQ ID NO: 66 - protein, 15 aa, John Cunningham polyomavirus
DAQVEEVRVFEGTEE

SEQ ID NO: 67 - protein, 10 aa, John Cunningham polyomavirus
QAVDTVAAKQ

SEQ ID NO: 68 - protein, 10 aa, John Cunningham polyomavirus
  MOD_RES (3)..(3): V or M
  MOD_RES (4)..(4): E, Q or G
MLXXRFNFLL

SEQ ID NO: 69 - protein, 9 aa, John Cunningham polyomavirus
LLLIWFRPV

SEQ ID NO: 70 - protein, 9 aa, John Cunningham polyomavirus
  MOD_RES (2)..(2): I or V
SXTEVECFL

SEQ ID NO: 71 - protein, 9 aa, John Cunningham polyomavirus
RLDLEISMY

SEQ ID NO: 72 - protein, 10 aa, John Cunningham polyomavirus
  MOD_RES (3)..(3): K or R
SVXVNLERKH

SEQ ID NO: 73 - protein, 9 aa, John Cunningham polyomavirus
  MOD_RES (5)..(5): K or R
AYLKXCKEL

SEQ ID NO: 74 - protein, 10 aa, John Cunningham polyomavirus
  MISC_FEATURE (1)..(1): May or may not be present
NLLMWEAVTV

SEQ ID NO: 75 - protein, 15 aa, John Cunningham polyomavirus
GSQQWRGLSRYFKVQ

SEQ ID NO: 76 - protein, 15 aa, John Cunningham polyomavirus
RGLSRYFKVQLRKRR

SEQ ID NO: 77 - protein, 15 aa, John Cunningham polyomavirus
RKAYLKKCKELHPDK

SEQ ID NO: 78 - protein, 15 aa, John Cunningham polyomavirus
WDEDLFCHEEMFASD

SEQ ID NO: 79 - protein, 15 aa, John Cunningham polyomavirus
CFRQWFGCDLTQEAL

SEQ ID NO: 80 - protein, 15 aa, John Cunningham polyomavirus
GGDEDKMKRMNFLYK

SEQ ID NO: 81 - protein, 15 aa, John Cunningham polyomavirus
KMKRMNFLYKKMEQG

SEQ ID NO: 82 - protein, 20 aa, John Cunningham polyomavirus
LNIPKKRYWLFKGPIDSGKT

SEQ ID NO: 83 - protein, 15 aa, John Cunningham polyomavirus
KRYWLFKGPIDSGKT

SEQ ID NO: 84 - protein, 15 aa, John Cunningham polyomavirus
VGPLCKGDNLYLSAV

SEQ ID NO: 85 - protein, 15 aa, John Cunningham polyomavirus
AYLDKNKAYPVECWV

SEQ ID NO: 86 - protein, 15 aa, John Cunningham polyomavirus
DMMRYVDRYGQLQTK

SEQ ID NO: 87 - protein, 10 aa, Artificial Sequence
  Description of Artificial Sequence Synthetic peptide
  MOD_RES (1)..(1): D or K
  MOD_RES (3)..(3): Q or K
  MOD_RES (9)..(9): K or R
  MISC_FEATURE (10)..(10): May or may not be present
  See specification as filed for detailed description of substitutions and preferred embodiments
XSXHSTPPXK

SEQ ID NO: 88 - protein, 9 aa, Artificial Sequence
  Description of Artificial Sequence Synthetic peptide
  MOD_RES (6)..(6): L or A
  MOD_RES (9)..(9): K or Q
AVDTVXAKX

SEQ ID NO: 89 - protein, 9 aa, Artificial Sequence
  Description of Artificial Sequence Synthetic peptide
  MOD_RES (4)..(4): I or F
  MOD_RES (8)..(8): T or R
CYCXDCFXQ

SEQ ID NO: 90 - protein, 9 aa, Artificial Sequence
  Description of Artificial Sequence Synthetic peptide
  MOD_RES (1)..(1): L or I
  MOD_RES (3)..(3): L or V
XPXMRKAYL

SEQ ID NO: 91 - protein, 9 aa, Artificial Sequence
  Description of Artificial Sequence Synthetic peptide
  MOD_RES (3)..(3): L or P
  MOD_RES (4)..(4): P or N
  MOD_RES (5)..(5): P or S
FPXXXDTLY

SEQ ID NO: 92 - protein, 9 aa, Artificial Sequence
  Description of Artificial Sequence Synthetic peptide
  MOD_RES (1)..(1): F or T
  MOD_RES (9)..(9): I or N
XLYCKEWPX

SEQ ID NO: 93 - protein, 9 aa, Artificial Sequence
  Description of Artificial Sequence Synthetic peptide
  MOD_RES (1)..(1): E or S
  MOD_RES (4)..(4): V or G
  MOD_RES (5)..(5): W or R
  MOD_RES (6)..(6): I or K
  See specification as filed for detailed description of substitutions and preferred embodiments
XPLXXXDCY

SEQ ID NO: 94 - protein, 10 aa, Artificial Sequence
  Description of Artificial Sequence Synthetic peptide
  MOD_RES (6)..(6): M or L
  MOD_RES (7)..(7): L or M
  MOD_RES (9)..(9): M or Q
VHCPCXXCXL

SEQ ID NO: 95 - protein, 9 aa, Artificial Sequence
  Description of Artificial Sequence Synthetic peptide
  MOD_RES (3)..(3): I or F
  MOD_RES (7)..(7): T or R
  MOD_RES (8)..(8): Q or E
YCXDCFXXW

SEQ ID NO: 96 - protein, 9 aa, Artificial Sequence
  Description of Artificial Sequence Synthetic peptide
  MOD_RES (3)..(3): L or M
  MOD_RES (8)..(8): E or D
LLXGMYLXF

SEQ ID NO: 97 - protein, 11 aa, Artificial Sequence
  Description of Artificial Sequence Synthetic peptide
  MOD_RES (2)..(2): F or L
  MOD_RES (10)..(10): E or D
VXLLMGMYLXF

SEQ ID NO: 98 - protein, 9 aa, Artificial Sequence
  Description of Artificial Sequence Synthetic peptide
  MOD_RES (1)..(1): I or V
  MOD_RES (3)..(3): E or G
  MOD_RES (6)..(6): Q or H
XEXSIXGGL

SEQ ID NO: 99 - protein, 10 aa, Artificial Sequence
  Description of Artificial Sequence Synthetic peptide
  MOD_RES (4)..(4): I, M or L
  MOD_RES (6)..(6): I or V
  MOD_RES (8)..(8): S or L
  MOD_RES (10)..(10): L or N
TEVXGXTXMX

SEQ ID NO: 100 - protein, 15 aa, Artificial Sequence
  Description of Artificial Sequence Synthetic peptide
  MOD_RES (8)..(8): I or M
  MOD_RES (11)..(11): I or V
  MOD_RES (13)..(13): R or K
  MOD_RES (14)..(14): Q or Y
LPGDPDMXRYXDXXG

SEQ ID NO: 101 - protein, 15 aa, Artificial Sequence
  Description of Artificial Sequence Synthetic peptide
  MOD_RES (2)..(2): I or V
  MOD_RES (5)..(5): L or M
  MOD_RES (7)..(7): T or I
  MOD_RES (9)..(9): S or R
  MOD_RES (12)..(12): T or S
DXCGXFXNXSGXQQW

SEQ ID NO: 102 - protein, 10 aa, Artificial Sequence
  Description of Artificial Sequence Synthetic peptide
  MOD_RES (7)..(7): L or A
  MOD_RES (10)..(10): K or Q
QAVDTVXAKX

SEQ ID NO: 103 - protein, 10 aa, Artificial Sequence
  Description of Artificial Sequence Synthetic peptide
  MOD_RES (3)..(3): T, V or M
  MOD_RES (4)..(4): E, D or Q
  MOD_RES (8)..(8): H or F
  MOD_RES (9)..(9): I or L
MLXXRFNXXL

SEQ ID NO: 104 - protein, 9 aa, Artificial Sequence
  Description of Artificial Sequence Synthetic peptide
  MOD_RES (1)..(1): A or S
  MOD_RES (2)..(2): I or V
  MOD_RES (4)..(4): E or Q
  See specification as filed for detailed description of substitutions and preferred embodiments
XXTXVECFL

SEQ ID NO: 105 - protein, 9 aa, Artificial Sequence
  Description of Artificial Sequence Synthetic peptide
  MOD_RES (1)..(1): R or K
  MOD_RES (4)..(4): S or L
XLDXEISMY

SEQ ID NO: 106 - protein, 10 aa, Artificial Sequence
  Description of Artificial Sequence Synthetic peptide
  MOD_RES (3)..(3): K or R
  MOD_RES (8)..(8): E or R
SVXVNLEXKH

SEQ ID NO: 107 - protein, 9 aa, Artificial Sequence
  Description of Artificial Sequence Synthetic peptide
  MOD_RES (4)..(4): R or K
  MOD_RES (9)..(9): F or L
AYLXKCKEX

SEQ ID NO: 108 - protein, 9 aa, Artificial Sequence
  Description of Artificial Sequence Synthetic peptide
  MISC_FEATURE (1)..(1): May or may not be present
  MOD_RES (2)..(2): I or V
  MOD_RES (9)..(9): L or V
NXMWEAVTX

SEQ ID NO: 109 - protein, 15 aa, Artificial Sequence
  Description of Artificial Sequence Synthetic peptide
  MOD_RES (2)..(2): T or S
  MOD_RES (9)..(9): A or S
  MOD_RES (14)..(14): I or V
  MOD_RES (15)..(15): R or Q
GXQQWRGLXRYFKXX

SEQ ID NO: 110 - protein, 15 aa, Artificial Sequence
  Description of Artificial Sequence Synthetic peptide
  MOD_RES (4)..(4): A or S
  MOD_RES (9)..(9): I or V
  MOD_RES (10)..(10): R or Q
  MOD_RES (15)..(15): S or R
RGLXRYFKXXLRKRX

SEQ ID NO: 111 - protein, 15 aa, Artificial Sequence
  Description of Artificial Sequence Synthetic peptide
  MOD_RES (6)..(6): R or K
  MOD_RES (7)..(7): R or K
  MOD_RES (11)..(11): F or L
  See specification as filed for detailed description of substitutions and preferred embodiments
RKAYLXXCKEXHPDK

SEQ ID NO: 112 - protein, 15 aa, Artificial Sequence
  Description of Artificial Sequence Synthetic peptide
  MOD_RES (10)..(10): D or E
WDEDLFCHEXMFASD

SEQ ID NO: 113 - protein, 15 aa, Artificial Sequence
  Description of Artificial Sequence Synthetic peptide
  MOD_RES (3)..(3): T or R
  MOD_RES (8)..(8): L or C
  MOD_RES (12)..(12): E or Q
  MOD_RES (14)..(14): T or A
CFXQWFGXDLTXEXL

SEQ ID NO: 114 - protein, 15 aa, Artificial Sequence
  Description of Artificial Sequence Synthetic peptide
  MOD_RES (12)..(12): T or F
GGDEDKMKRMNXLYK

SEQ ID NO: 115 - protein, 15 aa, Artificial Sequence
  Description of Artificial Sequence Synthetic peptide
  MOD_RES (7)..(7): T or F
  MOD_RES (15)..(15): D or G
KMKRMNXLYKKMEQX

SEQ ID NO: 116 - protein, 20 aa, Artificial Sequence
  Description of Artificial Sequence Synthetic peptide
  MOD_RES (1)..(1): F or L
  MOD_RES (3)..(3): V or I
  MOD_RES (6)..(6): R or K
XNXPKXRYWLFKGPIDSGKT

SEQ ID NO: 117 - protein, 15 aa, Artificial Sequence
  Description of Artificial Sequence Synthetic peptide
  MOD_RES (1)..(1): R or K
XRYWLFKGPIDSGKT

SEQ ID NO: 118 - protein, 15 aa, Artificial Sequence
  Description of Artificial Sequence Synthetic peptide
  MOD_RES (7)..(7): A or G
  MOD_RES (9)..(9): S or N
VGPLCKXDXLYLSAV

SEQ ID NO: 119 - protein, 15 aa, Artificial Sequence
  Description of Artificial Sequence Synthetic peptide
  MOD_RES (7)..(7): N or K
  MOD_RES (15)..(15): I or V
AYLDKNXAYPVECWX

SEQ ID NO: 120 - protein, 15 aa, Artificial Sequence
  Description of Artificial Sequence Synthetic peptide
  MOD_RES (3)..(3): I or M
  MOD_RES (6)..(6): I or V
  MOD_RES (9)..(9): Q or G
DMXRYXDRXGQLQTK

SEQ ID NO: 121 - protein, 9 aa, BK polyomavirus
SQHSTPPKK

SEQ ID NO: 122 - protein, 10 aa, BK polyomavirus
FPLCPDTLYC

SEQ ID NO: 123 - protein, 9 aa, BK polyomavirus
LLIKGGVEV

SEQ ID NO: 124 - protein, 9 aa, John Cunningham polyomavirus
SQHSTPPKK

SEQ ID NO: 125 - protein, 10 aa, John Cunningham polyomavirus
FPPNSDTLYC

SEQ ID NO: 126 - protein, 9 aa, John Cunningham polyomavirus
LLIKGGVEV

SEQ ID NO: 127 - protein, 15 aa, Artificial Sequence
  Description of Artificial Sequence Synthetic peptide
SVHCPCMLCQLRLRH

SEQ ID NO: 128 - protein, 14 aa, Artificial Sequence
  Description of Artificial Sequence Synthetic peptide
SVHCPCMLCQLRLR

SEQ ID NO: 129 - protein, 13 aa, Artificial Sequence
  Description of Artificial Sequence Synthetic peptide
SVHCPCMLCQLRL

SEQ ID NO: 130 - protein, 12 aa, Artificial Sequence
  Description of Artificial Sequence Synthetic peptide
SVHCPCMLCQLR

SEQ ID NO: 131 - protein, 11 aa, Artificial Sequence
  Description of Artificial Sequence Synthetic peptide
SVHCPCMLCQL

SEQ ID NO: 132 - protein, 10 aa, Artificial Sequence
  Description of Artificial Sequence Synthetic peptide
SVHCPCMLCQ

SEQ ID NO: 133 - protein, 9 aa, Artificial Sequence
  Description of Artificial Sequence Synthetic peptide
SVHCPCMLC

SEQ ID NO: 134 - protein, 14 aa, Artificial Sequence
  Description of Artificial Sequence Synthetic peptide
VHCPCMLCQLRLRH

SEQ ID NO: 135 - protein, 13 aa, Artificial Sequence
  Description of Artificial Sequence Synthetic peptide
HCPCMLCQLRLRH

SEQ ID NO: 136 - protein, 12 aa, Artificial Sequence
  Description of Artificial Sequence Synthetic peptide
CPCMLCQLRLRH

SEQ ID NO: 137 - protein, 11 aa, Artificial Sequence
  Description of Artificial Sequence Synthetic peptide
PCMLCQLRLRH

SEQ ID NO: 138 - protein, 10 aa, Artificial Sequence
  Description of Artificial Sequence Synthetic peptide
CMLCQLRLRH

SEQ ID NO: 139 - protein, 9 aa, Artificial Sequence
  Description of Artificial Sequence Synthetic peptide
MLCQLRLRH

SEQ ID NO: 140 - protein, 682 aa, BK polyomavirus
MDKVLNREESMELMDLLGLERAAWGNLPLMRKAYLRKCKEFHPDKGGDEDKMKRMNTLYKKMEQDVKVAHQPDFGTWSSSEVPTYGTEEWESWWSSFNEKWDEDLFCHEDMFASDEEATADSQHSTPPKKKRKVEDPKDFPSDLHQFLSQAVFSNRTLACFAVYTTKEKAILYKKLMEKYSVTFISRHMCAGHNIIFFLTPHRHRVSAINNFCQKLCTFSFLICKGVNKEYLLYSALTRDPYHIIEESIQGGLKEHDFNPEEPEETKQVSWKLITEYAVETKCEDVFLLLGMYLEFQYNVEECKKCQKKDQPYHFKYHEKHFANATIFAESKNQKSICQQAVDTVLAKKRVDTLHMTREEMLTERFNHILDKMDLIFGAHGNAVLEQYMAGVAWLHCLLPKMDSVIFDFLHCIVFNVPKRRYWLFKGPIDSGKTTLAAGLLDLCGGKALNVNLPMERLTFELGVAIDQYMVVFEDVKGTGAESKDLPSGHGINNLDSLRDYLDGSVKVNLEKKHLNKRTQIFPPGLVTMNEYPVPKTLQARFVRQIDFRPKIYLRKSLQNSEFLLEKRILQSGMTLLLLLIWFRPVADFADIQSRIVEWKERLDSEISMYTFSRMKYNICMGKCILDITREEDSETEDSGHGSSTESQSQCSSQVSDTSAPAEDSQRDPHSELHLCKGFQCF

SEQ ID NO: 141 - protein, 676 aa, John Cunningham polyomavirus
  MOD_RES (664)..(664): Any amino acid
MDKVLNREESMELMDLLGLDRSAWGNIPVMRKAYLKKCKELHPDKGGDEDKMKRMNFLYKKMEQGVKVAHQPDFGTWNSSEVPTYGTDEWESWWNTFNEKWDEDLFCHEEMFASDDENTGSQHSTPPKKKKKVEDPKDFPVDLHAFLSQAVFSNRTVASFAVYTTKEKAQILYKKLMEKYSVTFISRHGFGGHNILFFLTPHRHRVSAINNYCQKLCTFSFLICKGVNKEYLFYSALCRQPYAVVEESIQGGLKEHDFNPEEPEETKQVSWKLVTQYALTKCEDVFLLMGMYLDFQENPQCKKCEKKDQPNHFNHHEKHYYNAQIFADSKNQKSICQQAVDTVAAKQRVDSIHMTREEMLVERFNFLLDKMDLIFGAHGNAVLEQYMAGVAWIHCLLPQMDTVIYEFLKCIVLNIPKKRYWLFKGPIDSGKTTLAAALLDLCGGKSLNVNMPLERLNFELGVGIDQFMVVFEDVKGTGAESRDLPSGHGISNLDCLRDYLDGSVKVNLERKHQNKRTQVFPPGIVTMNEYSVPRTLQARFVRQIDFRPKAYLRKSLSCSEYLLEKRILQSGMTLLLLLIWFRPVADFAAAIHERIVQWKERLDLEISMYTFSTMKANVGMGRPILDFPREEDSEAEDSGHGSSTESQSQCSQVSEASGADTQEXCTYHICKGFQCF

SEQ ID NO: 142 - protein, 172 aa, BK polyomavirus
MDKVLNREESMELMDLLGLERAAWGNLPLMRKAYLRKCKEFHPDKGGDEDKMKRMNTLYKKMEQDVKVAHQPDFGTWSSSEVCADFPLCPDTLYCKEWPICSKKPSVHCPCMLCQLRLRHLNRKFLRKEPLVWIDCYCIDCFTQWFGLDLTEETLQWWVQIIGETPFRDLKL

SEQ ID NO: 143 - protein, 172 aa, John Cunningham polyomavirus
MDKVLNREESMELMDLLGLDRSAWGNIPVMRKAYLKKCKELHPDKGGDEDKMKRMNFLYKKMEQGVKVAHQPDFGTWNSSEVGCDFPPNSDTLYCKEWPNCATNPSVHCPCLMCMLKLRHRNRKFLRSSPLVWIDCYCFDCFRQWFGCDLTQEALHCWEKVLGDTPYRDLKL

SEQ ID NO: 144 - protein, 349 aa, BK polyomavirus
  MOD_RES (106)..(106): Any amino acid
  MOD_RES (128)..(128): Any amino acid
  MOD_RES (167)..(167): Any amino acid
  MOD_RES (272)..(272): Any amino acid
  MOD_RES (341)..(341): Any amino acid
MAPTKRKGECPGAAPKKPKPVQVPKLLIKGGVEVLEVKTGVDAITEVECFLNPEMGDPDLRGSLLEFSDSPRKMLPCYSTARIPLPNLNEDLTCGNLLMWEAVTVXTEVIGITSMLNLHAGSQKVHEXGGGKPIQGSNFHFFAVGGDPLEMQGVLMNYRTKYPEGTXTPKNPTAQSQVMNTDHKAYLDKNNAYPVECWIPDPSRNENTRYFGTTGGENVPPVLHVTNTATTVLLDEQGVGPLCKADSLYVSAADICGLFTNSSGTQQWRGLXRYFKIRLRKRSVKNPYPISFLLSDLINRRTQRVDGQPMYGMESQVEEVRVFDGTEQLPGDPDMIRYIDXQGQLQTKM

SEQ ID NO: 145 - protein, 353 aa, John Cunningham polyomavirus
  MOD_RES (117)..(117): Any amino acid
  MOD_RES (128)..(128): Any amino acid
  MOD_RES (158)..(158): Any amino acid
MAPTKRKGERKDPVQVPKLLIRGGVEVLEVKTGVDSITEVECFLTPEMGDPDEHLRGFSKSISISDTFESDSPNKDMLPCYSVARIPLPNLNEDLTCGNILMWEAVTLKTEVIGVTXLMNVHSNGQAXHDNGAGKPVQGTSFHFFSVGGEALELQGVXFNYRTKYPDGTIFPKNATVQSQVMNTEHKAYLDKNKAYPVECWVPDPTRNENTRYFGTLTGGENVPPVLHITNTATTVLLDEFGVGPLCKGDNLYLSAVDVCGMFTNRSGSQQWRGLSRYFKVQLRKRRVKNPYPISFLLTDLINRRTPRVDGQPMYGMDAQVEEVRVFEGTEELPGDPDMMRYVDRYGQLQTKM

SEQ ID NO: 146 - protein, 501 aa, BK polyomavirus
STPPKKKRKVEDPKDFPSDLHQFLSQAVFSNRTLACFAVYTTKEKAQILYKKLMEKYSVTFISRHMCAGHNIIFFLTPHRHRVSAINNFCQKLCTFSFLICKGVNKEYLLYSALTRDPYHTIEESIQGGLKEHDFSPEEPEETKQVSWKLITEYAVETKCEDVFLLLGMYLEFQYNVEECKKCQKKDQPYHFKYHEKHFANAIIFAESKNQKSICQQAVDTVLAKKRVDTLHMTREEMLTERFNHILDKMDLIFGAHGNAVLEQYMAGVAWLHCLLPKMDSVIFDFLHCIVFNVPKRRYWLFKGPIDSGKTTLAAGLLDLCGGKALNVNLPMERLTFELGVAIDQYMVVFEDVKGTGAESKDLPSGHGINNLDSLRDYLDGSVKVNLEKKHLNKRTQIFPPGLVTMNEYPVPKTLQARFVRQIDFRPKIYLRKSLQNSEFLLEKRILQSGMTLLLLLIWFRPVADFATDIQSRIVEWKERLDSEISMYTFSRMKYNICMGK

SEQ ID NO: 147 - protein, 493 aa, Merkel cell polyomavirus
STPPKPKKNRETPVPTDFPIDLSDYLSHAVYSNKTVSCFAIYTTSDKAIELYDKIEKFKVDFKSRHACELGCILLFITLSKHRVSAIKNFCSTFCTISFLICKGVNKMPEMYNNLCKPPYKLLQENKPLLNYEFQEKEKEASCNWNLVAEFACEYELDDHFIILAHYLDFAKPFPCQKCENRSRLKPHKAHEAHHSNAKLFYESKSQKTICQQAADTVLAKRRLEMLEMTRTEMLCKKFKKHLERLRDLDTIDLLYYMGGVAWYCCLFEEFEKKLQKIIQLLTENIPKYRNIWFKGPINSGKTSFAAALIDLLEGKALNINCPSDKLPFELGCALDKFMVVFEDVKGQNSLNKDLQPGQGINNLDNLRDHLDGAVAVSLEKKHVNKKHQIFPPCIVTANDYFIPKTLIARFSYTLHFSPKANLRDSLDQNMEIRKRRILQSGTTLLLCLIWCLPDTTFKPCLQEEIKNWKQILQSEISYGKFCQMIENVEAGQ

SEQ ID NO: 148 - protein, 96 aa, BK polyomavirus
MDKVLNREESMELMDLLGLERAAWGNLPLMRKAYLRKCKFFHPDKGGDEDKMKRMNTLYKKMEQDVKVAHQPDFGTWSSSEVPTYGTEEWESWWSS

SEQ ID NO: 149 - protein, 96 aa, Merkel cell polyomavirus
MDLVLNRKEREALCKLLEITPNCYGNIPLMKAAFKRSCLKHHPDKGGNPVIMMELNTLWSKFQQNIHKLRSDFSMFDEVDEAPIYGTTKFKEWWRS

SEQ ID NO: 150 - protein, 9 aa, BK polyomavirus
FKRPKTPPP

SEQ ID NO: 151 - protein, 9 aa, Merkel cell polyomavirus
FTTPKTPPP

SEQ ID NO: 152 - protein, 30 aa, BK polyomavirus
ETEDSGHGSSTESQSQCSSQVSDTSAPAED

SEQ ID NO: 153 - protein, 30 aa, Merkel cell polyomavirus
ETTNSGRESSTPNGTSVPRNSSRTDGTWED

SEQ ID NO: 154 - protein, 172 aa, BK polyomavirus; identical to SEQ ID NO: 142

SEQ ID NO: 155 - protein, 183 aa, Merkel cell polyomavirus
MDLVLNRKEREALCKLLEIAPNCYGNIPLMKAAFKRSCLKHHPDKGGNPVIMMELNTLWSKFQQNIHKLRSDFSMFDEVSTKFPWEEYGTLKDYMQSGYNARFCRGPGCMLKQLRDSKCACISCKLSRQHCSLKTLKQKNCLTWGECFCYQCFILWFGFPPTWESFDWWQKTLEETDYCLLHL

SEQ ID NO: 156 - protein, 359 aa, BK polyomavirus
MAPTKRKGECPGAAPKKPKEPVQVPKLLIKGGVEVLEVKTGVDAITEVECFLNPEMGDPDENLRGFSLKLSAENDFSSDSPERKMLPCYSTARIPLPNLNEDLTCGNLLMWEAVTVQTEVIGITSMLNLHAGSQKVHEHGGGKPIQGSNFHFFAVGGEPLEMQGVLMNYRSKYPDGTITPKNPTAQSQVMNTDHKAYLDKNNAYPVECWVPDPSRNENARYFGTFTGGENVPPVLHVTNTATTVLLDEQGVGPLCKADSLYVSAADICGLFTNSSGTQQWRGLARYFKIRLRKRSVKNPYPISFLLSDLINRRTQRVDGQPMYGMESQVEEVRVFDGTERLPGDPDMIRYIDKQGQLQT

SEQ ID NO: 157 - protein, 381 aa, Merkel cell polyomavirus
MAPKRKASSTCKTPKRQCIPKPGCCPNVASVPKLLVKGGVEVLSVVTGEDSITQIELYLNPRMGVNSPDLPTTSNWYTYTYDLQPKGSSPDQPIKENLPAYSVARVSLPMLNEDITCDTLQMWEAISVKTEVVGISSLINVHYWDMKRVHDYGAGIPVSGVNYHMFAIGGEPLDLQGLVLDYQTQYPKTTNGGPITIETVLGRKMTPKNQGLDPQAKAKLDKDGNYPIEVWCPDPSKNENSRYYGSIQTGSQTPTVLQFSNTLTTVLLDENGVGPLCKGDGLFISCADIVGFLFKTSGKMALHGLPRYFNVTLRKRWVKNPYPVVNLINSLFSNLMPKVSGQPMEGKDNQVEEVRIYEGSEQLPGDPDIVRFLDKFGQEKT

SEQ ID NO: 158 - protein, 9 aa, BK polyomavirus
EPLVWIDCY

SEQ ID NO: 159 - protein, 9 aa, BK polyomavirus
YCIDCFTQW

SEQ ID NO: 160 - protein, 11 aa, BK polyomavirus
VFLLLGMYLEF

SEQ ID NO: 161 - protein, 9 aa, BK polyomavirus
IEESIQGGL

SEQ ID NO: 162 - protein, 10 aa, BK polyomavirus
TEVIGITSML

SEQ ID NO: 163 - protein, 10 aa, BK polyomavirus
MLTERFNHIL

SEQ ID NO: 164 - protein, 9 aa, BK polyomavirus
AITEVECFL

SEQ ID NO: 165 - protein, 9 aa, BK polyomavirus
RLDSEISMY

SEQ ID NO: 166 - protein, 9 aa, BK polyomavirus
SVKVNLEKK

SEQ ID NO: 167 - protein, 9 aa, BK polyomavirus
AYLRKCKEF

SEQ ID NO: 168 - protein, 15 aa, BK polyomavirus
DICGLFTNSSGTQQW

SEQ ID NO: 169 - protein, 10 aa, BK polyomavirus
NLLMWEAVTV

SEQ ID NO: 170 - protein, 11 aa, John Cunningham polyomavirus
NREESMELMDL

SEQ ID NO: 171 - protein, 9 aa, John Cunningham polyomavirus
SPLVWIDCY

SEQ ID NO: 172 - protein, 9 aa, John Cunningham polyomavirus
LLIRGGVEV

SEQ ID NO: 173 - protein, 9 aa, John Cunningham polyomavirus
SITEVECFL

SEQ ID NO: 174 - protein, 10 aa, John Cunningham polyomavirus
NILMWEAVTL

SEQ ID NO: 175 - protein, 15 aa, John Cunningham polyomavirus
LNIPKKRYWLFKGPI